(19) United States
(12) Patent Application Publication
(10) Pub. No.: US 2024/0287493 A1
(43) Pub. Date: Aug. 29, 2024

(54) COMPOSITIONS AND METHODS FOR TREATING PGM1 DEFICIENCY

(71) Applicant: UNIVERSITY OF UTAH RESEARCH FOUNDATION, Salt Lake City, UT (US)

(72) Inventors: Kent Lai, Salt Lake City, UT (US); Bijina Balakrishnan, Salt Lake City, UT (US)

(21) Appl. No.: 18/573,350

(22) PCT Filed: Jun. 24, 2022
(86) PCT No.: PCT/US2022/034896

§ 371 Date: Dec. 21, 2023

Related U.S. Application Data
(60) Provisional application No. 63/214,566, filed on Jun. 24, 2021.

(51) Int. Cl. C12N 9/90; A61K 48/00; A61P 9/00; C12N 15/86

(52) U.S. Cl. CPC C12N 9/90; A61K 48/005; A61P 9/00; C12N 15/86; C12N 2750/14143; C12N 2800/22; C12N 2830/50; C12Y 504/02002

(57) ABSTRACT

Disclosed herein, are compositions and methods useful in expressing a functional PGM1 protein in a subject by administration of a recombinant adeno-associated virus vector containing a transgene encoding PGM1. Also disclosed herein are methods for treating a PGM1 gene deficiency in a subject in need thereof.

### CROSS REFERENCE TO RELATED APPLICATIONS

This application claims the benefit of U.S. Provisional Application No. 63/214,566, filed Jun. 24, 2021. The content of this earlier filed application is hereby incorporated by reference herein in their entirety.

### INCORPORATION OF THE SEQUENCE LISTING

The present application contains a sequence listing that is submitted via EFS-Web concurrent with the filing of this application, containing the file name “21101_0425P1_SL.txt” which is 32,768 bytes in size, created on Jun. 17, 2022, and is herein incorporated by reference in its entirety.

### BACKGROUND

Human phosphoglucomutase I (PGM1) deficiency is an ultra-rare genetic disorder in which the body cannot sustain cellular glucose homeostasis. In this disorder, the liver is unable to maintain normal glycemia and the muscles cannot use glucose as a viable energy source (Stiers et al. 2018, Structure 26, 1337-1345). Those affected by PGM1 exhibit multiple phenotypes including dilated cardiomyopathy, exercise intolerance, hepatopathy, hypoglycemia, muscle weakness, rhabdomyolysis, growth retardation, hypogonadotropic hypogonadism, and/or cleft palate (Schrapers et al. JIMD Rep. 2016; 26: 77-84). Currently, no cure for the disease exists. Additionally, because this disorder is so rare, limited data on the incidence of the condition or associated morbidity and mortality is available.

Although the Food and Drug Administration (FDA) has not approved any treatments for PGM1 deficiency, clinical studies have shown that dietary supplementation with d-galactose leads to improved protein glycosylation profiles (Voermans et al. Neuromuscular Disorders, Vol. 27, Issue 4, April 2017, pp. 370-376). These results have prompted clinical trials of d-galactose supplementation to study the effect on PGM1 patients. Data from the Phase II trial demonstrated that treatment with d-galactose improved growth, reduced hypoglycemic episodes, and recovered from hypogonadotropic hypogonadism. Additionally, the therapy normalized skeletal muscle substrate use during exercise, improved walking distance, and was safe and well tolerated. However, d-galactose treatment has been limited to being efficacious for a few of the 21 phenotypes of the disease; hence, there is a medical need for new treatments of the condition.

### SUMMARY

Disclosed herein are polynucleotides comprising an expression cassette, wherein the expression cassette comprises a transcriptional regulatory region comprising a promoter operatively linked to a nucleotide sequence as set forth in SEQ ID NO: 1 encoding the human phosphoglucomutase 1 (hPGM1) protein.

Disclosed herein are recombinant adeno-associated virus (AAV) vectors comprising an expression cassette comprising: a nucleic acid sequence encoding human phosphoglucomutase 1 (hPGM1), operably linked to one or more regulatory elements; and a polyadenylation tail signal.

Disclosed herein are recombinant adeno-associated virus (AAV) vectors comprising an AAV9 capsid containing a nucleic acid construct comprising a codon optimized nucleotide sequence encoding human phosphoglucomutase 1 (hPGM1) having the sequence set forth in of SEQ ID NO: 1 operably linked to regulatory elements.

Disclosed herein are isolated nucleic acid constructs comprising a codon optimized PGM1 encoding nucleotide sequence as set forth by SEQ ID NO: 1 operably linked to regulatory elements for expression of the PGM1 encoding nucleotide sequence in a subject.

### BRIEF DESCRIPTION OF THE DRAWINGS

FIG. 1 shows PGM1's role in multiple biological processes. PGM1 catalyzes the interconversion of glucose-1 phosphate and glucose-6 phosphate and therefore, plays a regulatory role in glycolysis, glycogenesis, glycogenolysis, and glycosylation.

FIGS. 2A-B show the construction of Pgm2 cKO mice. FIG. 2A shows the Easi-CRIPSR method of generating a floxed Pgm2 allele. The Easi-CRIPSR (Efficient additions with ssDNA inserts-CRISPR) method is a well-established and highly-efficient method to mediate targeted genomic knockins (PMID: 28511701). For this study, mouse embryos are injected with two preassembled guide RNA and Cas9 endonuclease ribonucleoprotein (RNP) complexes to create double stranded DNA breaks around exon 2 of Pgm2 (left/right cut sites). A long single-stranded DNA (IssDNA) 854 base pair donor engineered to contain a floxed version of exon 2 (grey triangles), flanked by 114- and 153-base pair left and right homology arms, respectively (blue) is co-injected to serve as a DNA repair template. The guide RNAs are designed to direct Cas9 mediated cutting immediately adjacent to each homology arm (5′ sgRNA: TGTGACAGTCCATCTGGGGA (SEQ ID NO: 12), 3′sgRNA: GAATCAGAGCCCAGGGCTTC (5′-CTTCGGGACCCGAGACTAAG; SEQ ID NO: 13)), which facilitates insertion of the lssDNA donor via homology directed repair (HDR). This results in replacement of the wild type Pgm2 exon2 with a floxed version. Insertion of the floxed allele is identified via simple PCR and restriction fragment length polymorphism (RFLP) analyses and founder animals with insertions are sequenced to confirm correctness. The University of Utah Mutation Generation and Detection Core and Transgenic and Gene Targeting Core for generating the conditional Pgm2 mouse line. FIG. 2B shows the Western blot analysis of heart tissues from Pgm2 cKO mice. Mice Pgm2^{fl/fl} mice were crossed to mice that harbored the alpha-MHC-MerCreMer (αMHC-MerCreMer) transgene, which has the mouse cardiac-specific alpha-myosin heavy chain promoter (aMHC or alpha-MHC; Myh6) directing the expression of a tamoxifen-inducible Cre recombinase (MerCreMer) in juvenile and adult cardiac myocytes. These aMHC-MerCreMer transgenic mice allow the creation of bi-transgenic mice for Cre-lox studies of temporally regulated deletion of loxP-flanked targeted genes in cardiac tissues/cells. Once the progeny with the desired combined genotypes were obtained and confirmed, oral treatment of the animals began with tamoxifen (35 mg/kg body weight for 5 days) at approximately 4 weeks of age in order to induce the excision of exon 2 of the Pgm2 alleles in the cardiomyocytes. Heart tissues were obtained from the Pgm2 cKO mice and subjected to Western blot analysis using an anti-PGM1 antibody.

FIGS. 3A-B show dilated heart and decreased cardiac functions in Pgm2 cKO mice. FIG. 3A shows the comparison of hearts from wild-type (WT) and Pgm2 KO mice 4 weeks after tamoxifen feeding N=6 for WT, N=5 for mutant. FIG. 3B shows the results of the echocardiography studies in WT and Pgm2 KO 12 weeks post-tamoxifen feeding. Data are mean±sem. N=3 for both WT and Mutant.

FIGS. 4A-B show the histochemical analysis of wild-type (WT), Pgm2 cKO mice and PGM1-CDG patient heart tissues. FIG. 4A show Masson's trichrome staining for fibrosis. FIG. 4B show PAS staining for glycogen.

FIGS. 5A-B show the ultra-structural analysis of wild-type (WT), Pgm2 cKO mice and PGM1-CDG patient heart tissues. Electron micrographs of low magnification (FIG. 5A) high magnification (FIG. 5B). Red arrows point to less organized mitochondrial cristae.

FIGS. 6A-D show AAV9-PGM1 gene replacement prevents manifestation of cardiac functions in the Pgm2 cKO mice. FIG. 6A shows a schematic of the overall experimental design. Cohorts (n=3) of 4-week-old male Pgm2^{fl/fl} mice were injected with 2.5E+13 vg/kg of AAV9-hPGM1 and were subsequently subjected to tamoxifen feeding two weeks later. Echocardiography was performed at regular time intervals after tamoxifen feeding and the data were compared to untreated Pgm2 cKO and wild type animals. FIG. 6B shows exemplified echocardiographs of wild-type, Pgm2 cKO and Pgm2 cKO mice treated with AAV9-hPGM1 at 24 weeks of age. FIG. 6C shows the quantified results of LV mass, ejection fraction, fractional shortening obtained from echocardiography at 8 and 24 weeks of age, respectively. FIG. 6D shows the histochemical studies of the heart tissues harvested from mice euthanized at 24 weeks.

FIGS. 7A-C show that AAV9-hPGM1 gene replacement halts/reverses the progression of cardiac functions in the Pgm2 cKO mice. FIG. 7A, left shows a schematic of the experimental design. Cohorts (n=3/4) of 4-week-old male Pgm2^{fl/fl} mice were fed with tamoxifen (35 mg/kg body weight for 5 days). Echography was performed two weeks later. Half of the cohorts were injected with 2.5E+13 vg/kg of AAV9-hPGM1 two weeks later. Echocardiography was performed at regular time intervals and the data were compared to untreated Pgm2 cKO and wild type animals. FIG. 7A, right shows a Western blot that show reconstitution of PGM1 protein in the hearts of the treated animals. FIG. 7B shows the quantified results of LV mass, ejection fraction, fractional shortening obtained from different cohorts upon echocardiography at different time points after AAV9-hPGM1 treatment. FIG. 7C shows the results of the histochemical studies of the heart tissues harvested from mice euthanized at 17 weeks after AAV9-hPGM1 therapy.

FIG. 8 shows a schematic of an example of an AAV vector gene therapy indicating the location of the PGM1 gene insertion.

### DETAILED DESCRIPTION

The present disclosure can be understood more readily by reference to the following detailed description of the invention, the figures and the examples included herein.

Before the present methods and compositions are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the term “transgene” refers to a gene or genetic material that has been transferred or artificially introduced into the genome by a genetic engineering technique from one organism to another, i.e., the host organism.

As used herein, the term “transgene expression” relates to the control of the amount and timing of appearance of the functional product of a transgene in a host organism.

The term “endogenous” as used herein refers to substances and processes originating from within an organism, tissue or cell.

“Inhibit,” “inhibiting” and “inhibition” mean to diminish or decrease gene expression, activity, response, condition, disease, or other biological parameter. This can include, but is not limited to, the complete ablation of the activity, response, condition, or disease. This may also include, for example, a 10% inhibition or reduction in gene expression, activity, response, condition, or disease as compared to the wild-type or control level. Thus, in some aspects, the inhibition or reduction can be a 10, 20, 30, 40, 50, 60, 70, 80, 90, 100%, or any amount of reduction in between as compared to native or control levels. In some aspects, the inhibition or reduction is 10-20, 20-30, 30-40, 40-50, 50-60, 60-70, 70-80, 80-90, or 90-100% as compared to wild-type or control levels. In some aspects, the inhibition or reduction is 0-25, 25-50, 50-75, or 75-100% as compared to wild-type or control levels.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the wild-type or control level. Thus, in some aspects, the increase or promotion can be a 10, 20, 30, 40, 50, 60, 70, 80, 90, 100%, or more, or any amount of promotion in between compared to native or control levels. In some aspects, the increase or promotion is 10-20, 20-30, 30-40, 40-50, 50-60, 60-70, 70-80, 80-90, or 90-100% as compared to wild-type or control levels. In some aspects, the increase or promotion is 0-25, 25-50, 50-75, or 75-100%, or more, such as, for example, 200, 300, 500, or 1000% more as compared to wild-type or control levels. In some aspects, the increase or promotion can be greater than 100 percent as compared to wild-type or control levels, such as 100, 150, 200, 250, 300, 350, 400, 450, 500% or more as compared to the wild-type or control levels.

The term “operatively linked to” refers to the functional relationship of a nucleic acid with another nucleic acid sequence. Promoters, enhancers, transcriptional and translational stop sites, and other signal sequences are examples of nucleic acid sequences linked to other sequences in order confer functional activity of the construct as a whole. For example, operative linkage of DNA to a transcriptional control element refers to the physical and functional relationship between the DNA and promoter such that the transcription of such DNA is initiated from the promoter by an RNA polymerase that specifically recognizes, binds to and transcribes the DNA.

As used herein, the terms “promoter,” “promoter element,” or “promoter sequence” are equivalents and as used herein, refers to a DNA sequence which when operatively linked to a nucleotide sequence of interest is capable of controlling the transcription of the nucleotide sequence of interest into mRNA. A promoter is located 5′ (i.e., upstream) of a nucleotide sequence of interest (e.g., proximal to the transcriptional start site of a structural gene), although not necessarily immediately upstream because of the optional inclusion of intervening sequences between the promoter and the sequence to be transcribed, whose transcription into mRNA it controls, and provides a site for specific binding by RNA polymerase and other transcription factors for initiation of transcription.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, the term “subject” refers to the target of administration, e.g., a human. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment for a PGM1 deficiency, such as, for example, prior to the administering step.

As used herein, the term “comprising” can include the aspects “consisting of” and “consisting essentially of.”

As used herein, the term “normal” refers to an individual, a sample or a subject that does not have PGM1 deficiency or does not have an increased susceptibility of developing PGM1 deficiency.

As used herein, the term “treat” or “treatment” refers to the medical management of a patient with the intent to cure, ameliorate, stabilize, (e.g., PGM1 deficiency). This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) inhibiting the disease, i.e., arresting its development; or (ii) relieving the disease, i.e., causing regression of the disease (e.g., PGM1 deficiency).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed. For example, “prevent” is meant to mean minimize the chance that a subject who has an increased susceptibility for developing PGM1 deficiency will develop PGM1 deficiency. In the context as used herein, preventing does not need to eliminate completely all sequele associated with PGM1 deficiency and would encompass any reduction in the expression of one or more symptoms associated or disease conditions associated with PGM1 deficiency.

The term “vector” or “construct” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

The term “expression vector” is herein to refer to vectors that are capable of directing the expression of genes to which they are operatively-linked. Common expression vectors of utility in recombinant DNA techniques are often in the form of plasmids. Recombinant expression vectors can comprise a nucleic acid as disclosed herein in a form suitable for expression of the acid in a host cell. In other words, the recombinant expression vectors can include one or more regulatory elements or promoters, which can be selected based on the host cells used for expression that is operatively linked to the nucleic acid sequence to be expressed.

“Modulate”, “modulating” and “modulation” as used herein mean a change in activity or function or number. The change may be an increase or a decrease, an enhancement or an inhibition of the activity, function or number.

The terms “alter” or “modulate” can be used interchangeable herein referring, for example, to the expression of a nucleotide sequence in a cell means that the level of expression of the nucleotide sequence in a cell after applying a method as described herein is different from its expression in the cell before applying the method.

As used herein, the terms “disease” or “disorder” or “condition” are used interchangeably referring to any alternation in state of the body or of some of the organs, interrupting or disturbing the performance of the functions and/or causing symptoms such as discomfort, dysfunction, distress, or even death to the person afflicted or those in contact with a person. A disease or disorder or condition can also related to a distemper, ailing, ailment, malady, disorder, sickness, illness, complaint, or affection.

Suitable promoters can be derived from genes of the host cells where expression should occur or from pathogens for this host cells (e.g., tissue promoters or pathogens like viruses). If a promoter is an inducible promoter, then the rate of transcription increases in response to an inducing agent. In contrast, the rate of transcription is not regulated by an inducing agent if the promoter is a constitutive promoter. Also, the promoter may be regulated in a tissue-specific or tissue preferred manner such that it is only active in transcribing the associated coding region in a specific tissue type(s) such as leaves, roots or meristem. The term “tissue specific” as it applies to a promoter refers to a promoter that is capable of directing selective expression of a nucleotide sequence or gene of interest to a specific type of tissue in the relative absence of expression of the same nucleotide sequence or gene of interest in a different type of tissue.

Although the foregoing invention has been described in some detail by way of illustration and example for purposes of clarity of understanding, certain changes and modifications may be practiced within the scope of the appended claims.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Human PGM1 deficiency is a debilitating, and potentially lethal genetic disorder. Currently, no cure for the disease is available and dietary management is insufficient. While dietary galactose supplementation may help relieve some biochemical phenotypes, it cannot fully correct all disease phenotypes, including the lethal cardiomyopathy. Also, long-term galactose supplementation in the patients may be harmful. Disclosed herein are compositions and methods that address the missing PGM1 activity in PGM1-deficient cells using an AAV9-gene therapy approach.

PGMI-CDG is an autosomal recessive disorder caused by a deficiency of Phosphoglucomutase 1, and was originally classified as a disease of glycogen metabolism, but later reclassified as a congenital disorder of glycosylation. PGM1-CDG presents as two major phenotypes: a primary myopathic one and a multisystem one. Most common clinical findings in PGM1-CDG include but are not limited to rhabdomyolysis (74%), hypoglycemia (67%), congenital malformation (44-49%), liver abnormalities (74%), exercise intolerance (35%), and dilated cardiomyopathy (21%). The pathophysiology of some tissue-specific disease phenotypes are unclear. Galactose therapy is less effective in cardiac and myopathy phenotypes.

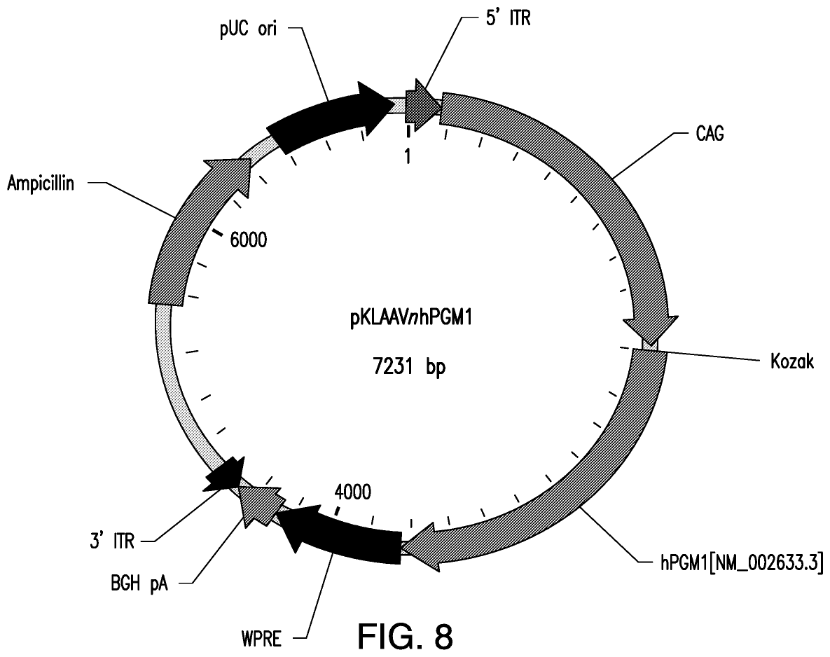

Disclosed herein are methods and compositions of a gene therapy that can insert the PGM1 gene and protein in those suffering from PGM1 deficiency, including patients that are heterozygous for the gene mutation. In some aspects, the therapeutic comprises an adeno-associated viral (e.g., AAV-9) vector, a viral-based gene delivery platform that can infect humans and deliver a gene to specific cells without causing disease. Therapeutics using AAV viral vectors are well-tolerated, and have achieved regulatory approval worldwide (Naso et al. BioDrugs, 2017; 31(4): 317-334). FIG. 8 shows a schematic of a gene therapy disclosed herein, where, for example, PGM1 is inserted between 1932 and 3620 regions (green) of the mammalian AAV vector. The nucleic acid construct also includes enhancements to the promotor region (pink) and other areas (purple) to enhance stability.

In some aspects, the compositions and methods disclosed herein can be used as a treatment or cure for PGM1 deficiency in humans. Also, the compositions and methods disclosed herein can be used in a subset of patients who do not respond to supplements such as d-galactose, and have an improved safety profile. Further, the compositions and methods disclosed herein can be used to treat or manage one or more of phenotypes of PGM1 deficiency and allow patients to live normal lives and reduce or eliminate disease-associated mortality.

### Compositions

Nucleic acids and polynucleotides. Disclosed herein are nucleic acids comprising at least one transgene operably linked to a promoter, wherein the transgene encodes PGM1 (Phosphoglucomutase 1; NM_002633.3). Also disclosed herein are polynucleotides comprising a transcriptional regulatory region comprising a promoter operatively linked to a nucleotide sequence as set forth in SEQ ID NO: 1 encoding the human phosphoglucomutase 1 (hPGM1) protein. The PGM1 gene encodes Phosphoglucomutase 1, an enzyme involved in glycolysis and gluconeogenesis.

The PGM1 gene can encode an mRNA having the nucleotide sequence of NM_002633.3. The PGM1 gene can encode a protein having the amino acid sequence NP_002624.2. In some aspects, the PGM1 gene is codon-optimized, for example, for expression in a mammal, such as a human. Sequences corresponding to all GenBank accession numbers described in the disclosure are incorporated herein by reference in their entirety. Note that DNA sequences provided herein may also include the reverse complement to form the double stranded DNA sequence or may be a reverse complement of the sequences disclosed herein.

In some aspects, hPGMI has the amino acid sequence as follows:

(SEQ ID NO: 11)
MVKIVTVKTQAYQDQKPGTSGLRKRVKVFQSSANYAENFIQSIISTVEP
AQRQEATLVVGGDGRFYMKEAIQLIARIAAANGIGRLVIGQNGILSTPA
VSCIIRKIKAIGGIILTASHNPGGPNGDFGIKFNISNGGPAPEAITDKI
FQISKTIEEYAVCPDLKVDLGVLGKQQFDLENKFKPFTVEIVDSVEAYA
TMLRSIFDFSALKELLSGPNRLKIRIDAMHGVVGPYVKKILCEELGAPA
NSAVNCVPLEDFGGHHPDPNLTYAADLVETMKSGEHDFGAAFDGDGDRN
MILGKHGFFVNPSDSVAVIAANIFSIPYFQQTGVRGFARSMPTSGALDR
VASATKIALYETPTGWKFFGNLMDASKLSLCGEESFGTGSDHIREKDGL
WAVLAWLSILATRKQSVEDILKDHWQKYGRNFFTRYDYEEVEAEGANKM
MKDLEALMFDRSFVGKQFSANDKVYTVEKADNFEYSDPVDGSISRNQGL
RLIFTDGSRIVFRLSGTGSAGATIRLYIDSYEKDVAKINQDPQVMLAPL
ISIALKVSQLQERTGRTAPTVIT.

In some aspects, the nucleic acid sequence encoding PGM1 can be SEQ ID NO: 1. In some aspects, the nucleic acid sequence encoding PGM1 comprises at least 80%, at least 85%, at least 90%, at least 95%, at least 96%, at least 97%, at least 98%, or at least 99% identity to SEQ ID NO: 1. In some aspects, the nucleic acid sequence encoding PGM1 gene comprises up to 20 nucleotides that are different from the PGM1 gene set forth in SEQ ID NO: 1. In some aspects, the PGM1 gene comprises 1, 2, 3, 4, 5, 6, 7, 8, 9, 10, 11, 12, 13, 14, 15, 16, 17, 18, 19, or 20 nucleotides that are different from the PGM1 gene set forth in SEQ ID NO: 1. In some aspects, the nucleic acid sequence encoding PGM1 gene comprises more than 20 nucleotides that are different from the PGM1 gene set forth in SEQ ID NO: 1.

In some aspects, the nucleic acid sequence encoding PGM1 comprises insertions relative to SEQ ID NO: 1. In some aspects, the nucleic acid sequences encoding PGM1 comprises insertions relative to SEQ ID NO: 1 that do not introduce a frameshift mutation. In some aspects, an insertion in the nucleic acid sequence relative to SEQ ID NO: 1 involves the insertion of multiples of 3 nucleotides (e.g., 3, 6, 9, 12, 15, 18, etc.). In some aspects, an insertion in the nucleic acid sequence relative to SEQ ID NO: 1 leads to an increase in the total number of amino acid residues in the resultant PGM1 protein (e.g., an increase of 1-3, 15, 3-10, 5-10, 5-15, or 10-20 amino acid residues).

In some aspects, the nucleic acid sequence encoding PGM1 comprises deletions relative to SEQ ID NO: 1. In some aspects, the nucleic acid sequences encoding PGM1 comprises deletions relative to SEQ ID NO: 1 that do not introduce a frameshift mutation. In some aspects, a deletion in the nucleic acid sequence relative to SEQ ID NO: 1 involves the deletion of multiples of 3 nucleotides (e.g., 3, 6, 9, 12, 15, 18, etc.). In some aspects, a deletion in the nucleic acid sequence relative to SEQ ID NO: 1 leads to an decrease in the total number of amino acid residues in the resultant PGM1 protein (e.g., a decrease of 1-3, 1-5, 3-10, 5-10, 5-15, or 10-20 amino acid residues).

In some aspects, the nucleic acid sequence encoding PGM1 can be a codon-optimized sequence (e.g., codon optimized for expression in mammalian cells). In some aspects, a codon-optimized sequence encoding PGM1 comprises reduced GC content relative to a wild-type sequence that has not been codon-optimized. In some aspects, a codon-optimized sequence encoding PGM1 comprises a 1-5%, 3-5%, 3-10%, 5-10%, 5-15%, 10-20%, 15-30%, 20-40%, 25-50%, or 30-60% reduction in GC content relative to a wild-type sequence that has not been codon-optimized. In some aspects, a codon-optimized sequence encoding PGM1 comprises fewer guanine and/or cytosine nucleobases relative to a wild-type sequence that has not been codon-optimized. In some aspects, a codon-optimized sequence encoding PGM1 comprises 1-5, 3-5, 3-10, 5-10, 5-15, 10-20, 15-30, 20-40, 25-50, or 30-60 fewer guanine and/or cytosine nucleobases relative to a wild-type sequence that has not been codon-optimized. In some aspects, a codon-optimized sequence encoding PGM1 comprises fewer CpG dinucleotide islands relative to a wild-type sequence that has not been codon-optimized. In some aspects, a codon-optimized sequence encoding PGM1 comprises 1-3, 3-5, 3-10, 5-10, 5-15, 10-20, 15-30, 20-40, 25-50, or 30-60 fewer CpG dinucleotide islands relative to a wild-type sequence that has not been codon-optimized. In some aspects, the nucleotide sequence encoding PGM1 is SEQ ID NO: 1.

Promoters. In the constructs disclosed herein nucleic acid encoding the PGM1 protein, including, the nucleotide sequence of SEQ ID NO: 1, can be operably linked to a promoter to direct expression of the PGM1 coding sequence, particularly in cardiac muscle cells. In some aspects, the promoter can be a constitutive promoter. In some aspects, the promoter can be a constitutive promoter, for example a CAG promoter, a chicken beta-actin (CBA) promoter, a retroviral Rous sarcoma virus (RSV) LTR promoter (optionally with the RSV enhancer), a cytomegalovirus (CMV) promoter (optionally with the CMV enhancer) [see, e.g., Boshart et al., Cell, 41:521-530 (1985)], a CMV enhanced chicken β-actin promoter (CB), a SV40 promoter, a dihydrofolate reductase promoter, a β-actin promoter, a phosphoglycerol kinase (PGK) promoter, or an EFla promoter [Invitrogen]. In some aspects, a promoter can be a CAG promoter. In some aspects, a promoter can be an enhanced chicken β-actin promoter. In some aspects, a promoter can be a U6 promoter. In some aspects, the promoter can be a CB6 promoter. In some aspects, the promoter can be a JeT promoter. In some aspects, a promoter can be a CB promoter. In some aspects, the CAG promoter has the sequence of SEQ ID NO: 2.

In some aspects, a promoter can be an inducible promoter. Inducible promoters allow regulation of gene expression and can be regulated by exogenously supplied compounds, environmental factors such as temperature, or the presence of a specific physiological state, e.g., acute phase, a particular differentiation state of the cell, or in replicating cells. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which can be useful include those which are regulated by a specific physiological state, e.g., temperature, acute phase, a particular differentiation state of the cell, or in replicating cells.

In some aspects, the native promoter for the transgene (e.g., PGM1) can be used. In some aspects, the native promoter can be used when it is desired that expression of the transgene should mimic the expression of a native wild-type PGM1 gene (e.g., a non-mutated PGM1 gene). The native promoter can be used when expression of the transgene must be regulated temporally or developmentally, or in a tissue-specific manner, or in response to specific transcriptional stimuli. In some aspects, other native expression control elements, such as enhancer elements, polyadenylation sites or Kozak consensus sequences can also be used to mimic the native expression. In some aspects, the promoter can drive transgene expression in a non-tissue specific manner. In some aspects, the promoter can drives transgene expression in a specific tissue.

In some aspects, the promoter drives transgene expression in heart tissues (e.g., cardiomyocytes). In some aspects, the promoter drives transgene expression in liver tissue. In some aspects, the promoter drives transgene expression brain tissue. In some aspects, the promoter drives transgene expression in skeletal muscle tissue. In some aspects, the promoter drives transgene expression heart tissue, liver tissue, brain tissue, skeletal muscle tissue or a combination thereof. In some aspects, the disclosure provides a nucleic acid operably comprising a tissue-specific promoter operably linked to a transgene. As used herein, “tissue-specific promoter” refers to a promoter that preferentially regulates (e.g., drives or up-regulates) gene expression in a particular cell type relative to other cell types. A cell-type-specific promoter can be specific for any cell type, such as central nervous system (CNS) cells, liver cells (e.g., hepatocytes), heart cells, muscle cells, etc. Examples of tissue-specific promoters include but are not limited to a liver-specific thyroxin binding globulin (TBG) promoter, an insulin promoter, a creatine kinase (MCK) promoter, a a-myosin heavy chain (a-MHC) promoter, or a cardiac Troponin T (cTnT) promoter. Other exemplary promoters include Beta-actin promoter, hepatitis B virus core promoter (Sandig et al., Gene Ther., 3:1002-9 (1996)); alpha-fetoprotein (AFP) promoter (Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998)), and the immunoglobulin heavy chain promoter.

As used herein, the term “hybrid promoter” refers to a regulatory construct capable of driving transcription of an RNA transcript (e.g., a transcript comprising encoded by a transgene) in which the construct comprises two or more regulatory elements artificially arranged. Typically, a hybrid promoter comprises at least one element that is a minimal promoter and at least one element having an enhancer sequence or an intronic, exonic, or UTR sequence comprising one or more transcriptional regulatory elements. In some aspects in which a hybrid promoter comprises an exonic, intronic, or UTR sequence, such sequence(s) can encode upstream portions of the RNA transcript while also containing regulatory elements that modulate (e.g., enhance) transcription of the transcript. In some aspects, two or more elements of a hybrid promoter can be from heterologous sources relative to one another. In some aspects, a hybrid promoter comprises a first sequence from the chicken beta-actin promoter and a second sequence of the CMV enhancer. In some aspects, the hybrid promoter comprises a first sequence from the CMV enhancer and a second sequence from the chicken beta-actin promoter. In some aspects, a hybrid promoter comprises a first sequence from a chicken beta-actin promoter and a second sequence from an intron of a chicken-beta actin gene. In some aspects, a hybrid promoter comprises a first sequence from the chicken beta-actin promoter fused to a CMV enhancer sequence and a sequence from an intron of the chicken-beta actin gene. In some aspects, a hybrid promoter comprises a CB6 promoter. In some aspects, a hybrid promoter comprises a JeT promoter. In some aspects, the promoter can be a CAG promoter. In some aspects, the CAG promoter comprises a CMV enhancer sequence and a CB promoter sequence. In some aspects, the enhancer sequence can be woodchuck hepatitis virus posttranscriptional regulatory element (WPRE). In some aspects, the WPRE sequence can be SEQ ID NO: 7.

In some aspects, the PGM1 expressing constructs disclosed herein, the PGMI coding sequence, for example, SEQ ID NO: 1, can be operably linked to the CAG “promoter” or regulatory sequence, which is SEQ ID NO: 2. In some aspects, the CAG promoter has a nucleotide sequence of SEQ ID NO: 2.

In some aspects, the vector can further comprise conventional control elements which are operably linked with elements of the transgene in a manner that permits its transcription, translation and/or expression in a cell transfected with the vector or infected with the virus produced by the disclosure. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (e.g., Kozak consensus sequence); sequences that enhance protein stability; and when desired, sequences that enhance secretion of the encoded product. A number of expression control sequences, including promoters which are native, constitutive, inducible and/or tissue-specific, are known in the art and may be utilized.

In some aspects, the constructs comprising the nucleotide sequence encoding PGM1 include an intron sequence which is operably linked and 5′ to the coding sequence. In particular, the intron sequence can be a chimeric intron. In some aspects, a chimeric intron comprises a nucleic acid sequence from a chicken beta-actin gene, for example a non-coding intronic sequence from intron 1 of the chicken beta-actin gene. In some aspects, the intronic sequence of the chicken beta-actin gene ranges from about 50 to about 150 nucleotides in length (e.g., any length between 50 and 150 nucleotides, inclusive). In some aspects, the intronic sequence of the chicken beta-actin gene ranges from about 100 to 120 (e.g., 100, 101, 102, 103, 104, 105, 106, 107, 108, 109, 110, 111, 112, 113, 114, 115, 116, 117, 118, 119, or 120) nucleotides in length. In some aspects, a chimeric intron can be adjacent to one or more untranslated sequences (e.g., an untranslated sequence located between the promoter sequence and the chimeric intron sequence and/or an untranslated sequence located between the chimeric intron and the first codon of the transgene sequence). In some aspects, each of the one or more untranslated sequences can be non-coding sequences from a bovine growth hormone gene (e.g., untranslated sequence from bovine growth hormone exon 1, exon 2, etc.). In some aspects, the intron sequence is the sequence of SEQ ID NO: 3 (which is one strand of the DNA sequence and may include the reverse complement as well forming the double stranded sequence).

In some aspects, the recombinant AAV vector comprises a posttranscriptional response element. As used herein, the term “posttranscriptional response element” refers to a nucleic acid sequence that, when transcribed, adopts a tertiary structure that enhances expression of a gene. Examples of posttranscriptional regulatory elements include, but are not limited to, woodchuck hepatitis virus posttranscriptional regulatory element (WPRE), mouse RNA transport element (RTE), constitutive transport element (CTE) of the simian retrovirus type 1 (SRV-1), the CTE from the Mason-Pfizer monkey virus (MPMV), and the 5′ untranslated region of the human heat shock protein 70 (Hsp70 5′UTR). In some aspects, the recombinant AAV vector comprises a woodchuck hepatitis virus posttranscriptional regulatory element (WPRE). In some aspects, the WPRE can be a mutant WRPE. In some aspects, the WPRE has or comprises SEQ ID NO: 7.

In some aspects, a polyadenylation sequence can be inserted following the transgene sequences and optionally before a 3′ AAV ITR sequence. A recombinant AAV construct useful in the disclosure can also contain an intron, desirably located between the promoter/enhancer sequence and the transgene. In some aspects, the polyA signal sequence can be a bovine growth hormone poly A sequence having the nucleotide sequence of SEQ ID NO: 3.

In some aspects, the gene expression cassette construct comprises or consists of elements arranged as follows:

CAG promoter-Kozak sequence-PGM1 coding sequence (SEQ ID NO: 1)-WPRE-bovine growth hormone PolyA signal sequence. The construct is depicted in FIG. 8. The nucleotide sequence of the gene expression cassette from CAG promoter to polyA signal sequence can be SEQ ID NO: 9.

In some aspects, the expression cassette can be flanked by adeno-associated virus inverted terminal repeats.

Recombinant AA Vs. In some aspects, the isolated nucleic acids disclosed herein can be recombinant adeno-associated viruses (rAAVs) vectors. In some aspects, the rAAV vectors described herein can be composed of, at a minimum, a transgene and its regulatory sequences, and 5′ and 3′ AAV inverted terminal repeats (ITRs). It is this recombinant AAV vector that can be packaged into a capsid protein and delivered to a selected target cell. In some aspects, the transgene can be a nucleic acid sequence, heterologous to the vector sequences, which encodes a polypeptide, protein, functional RNA molecule or other gene product, of interest. In some aspects, the nucleic acid coding sequence can be operatively linked to regulatory components in a manner which permits transgene transcription, translation, and/or expression in a cell of a target tissue.

Adeno-associated virus (AAV) is a replication-deficient parvovirus, the single-stranded DNA genome of which is about 4.7 kb in length including 145 nucleotide inverted terminal repeat (ITRs). There are multiple serotypes of AAV. The nucleotide sequences of the genomes of the AAV serotypes are known. For example, the nucleotide sequence of the AAV serotype 2 (AAV2) genome is presented in Srivastava et al., J Virol, 45: 555-564 (1983) as corrected by Ruffing et al., J Gen Virol, 75: 3385-3392 (1994). As other examples, the complete genome of AAV-1 is provided in GenBank Accession No. NC_002077; the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively (see also U.S. Pat. Nos. 7,282,199 and 7,790,449 relating to AAV-8); the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). Cloning of the AAVrh.74 serotype is described in Rodino-Klapac., et al. Journal of Translational Medicine 5, 45 (2007). Cis-acting sequences directing viral DNA replication (rep), encapsidation/packaging and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (e.g., at AAV2 nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. The rep and cap proteins may be provided in trans. Another significant feature of AAV is that it is an extremely stable and hearty virus. It easily withstands the conditions used to inactivate adenovirus (56.degree. C. to 65.degree. C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. See, Clark et al., Hum Gene Ther, 8: 659-669 (1997); Kessler et al., Proc Nat. Acad Sc. USA, 93: 14082-14087 (1996); and Xiao et al., J Virol, 70: 8098-8108 (1996). See also, Chao et al., Mol Ther, 2: 619-623 (2000) and Chao et al., Mol Ther, 4: 217-222 (2001). Moreover, because muscle is highly vascularized, recombinant AAV transduction has resulted in the appearance of transgene products in the systemic circulation following intramuscular injection as described in Herzog et al., Proc Natl Acad Sci USA, 94: 5804-5809 (1997) and Murphy et al., Proc Natl Acad Sci USA, 94: 13921-13926 (1997). Moreover, Lewis et al., J Virol, 76: 8769-8775 (2002) demonstrated that skeletal myofibers possess the necessary cellular factors for correct antibody glycosylation, folding, and secretion, indicating that muscle is capable of stable expression of secreted protein therapeutics.

As used herein, the term “AAV” is a standard abbreviation for adeno-associated virus. Adeno-associated virus is a single-stranded DNA parvovirus that grows only in cells in which certain functions are provided by a co-infecting helper virus. There are currently thirteen serotypes of AAV that have been characterized. General information and reviews of AAV can be found in, for example, Carter, 1989, Handbook of Parvoviruses, Vol. 1, pp. 169-228, and Berns, 1990, Virology, pp. 1743-1764, Raven Press, (New York). However, it is fully expected that these same principles will be applicable to additional AAV serotypes since it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

An “AAV vector” as used herein refers to a vector comprising one or more polynucleotides of interest (or transgenes) that are flanked by AAV terminal repeat sequences (ITRs). Such AAV vectors can be replicated and packaged into infectious viral particles when present in a host cell that has been transfected with a vector encoding and expressing rep and cap gene products.

An “AAV virion” or “AAV viral particle” or “AAV vector particle” refers to a viral particle composed of at least one AAV capsid protein and an encapsidated polynucleotide AAV vector. If the particle comprises a heterologous polynucleotide (i.e., a polynucleotide other than a wild-type AAV genome such as a transgene to be delivered to a mammalian cell), it is typically referred to as an “AAV vector particle” or simply an “AAV vector”. Thus, production of AAV vector particle necessarily includes production of AAV vector; as such a vector is contained within an AAV vector particle.

Recombinant AAV genomes of the invention comprise nucleic acid molecule of the invention and one or more AAV ITRs flanking a nucleic acid molecule. AAV DNA in the rAAV genomes may be from any AAV serotype for which a recombinant virus can be derived including, but not limited to, AAV serotypes AAVrh.74, AAV-1, AAV-2, AAV-3, AAV-4, AAV-5, AAV-6, AAV-7, AAV-8, AAV-9, AAV-10, AAV-11, AAV-12 and AAV-13. Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). As noted in the Background section above, the nucleotide sequences of the genomes of various AAV serotypes are known in the art. To promote skeletal muscle-specific expression, AAV1, AAV6, AAV8 or AAVrh.74 may be used.

DNA plasmids of the invention comprise rAAV genomes of the invention. The DNA plasmids are transferred to cells permissible for infection with a helper virus of AAV (e.g., adenovirus, El-deleted adenovirus or herpesvirus) for assembly of the rAAV genome into infectious viral particles. Techniques to produce rAAV particles, in which an AAV genome to be packaged, rep and cap genes, and helper virus functions are provided to a cell, are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs, including, but not limited to, AAV serotypes AAV-1, AAV-2, AAV-3, AAV-4, AAV-5, AAV-6, AAV-7, AAVrh.74, AAV-8, AAV-9, AAV-10, AAV-11, AAV-12 and AAV-13. Production of pseudotyped rAAV is disclosed in, for example, WO 01/83692 which is incorporated by reference herein in its entirety.

Methods of generating a packaging cell comprise creating a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79: 2077-2081), addition of synthetic linkers containing restriction endonuclease cleavage sites (Laughlin et al., 1983, Gene, 23: 65-73) or by direct, blunt-end ligation (Senapathy & Carter, 1984, J. Biol. Chem., 259: 4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial. and Immunol., 158: 97-129). Various approaches are described in Ratschin et al., Mol. Cell. Biol. 4: 2072 (1984); Hermonat et al., Proc. Natl. Acad. Sci. USA, 81: 6466 (1984); Tratschin et al., Mol. Cell. Biol. 5: 3251 (1985); McLaughlin et al., J. Virol., 62: 1963 (1988); and Lebkowski et al., Mol. Cell. Biol., 7: 349 (1988). Samulski et al., J. Virol., 63: 3822-3828 (1989); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. Vaccine 13: 1244-1250 (1995); Paul et al. Human Gene Therapy 4: 609-615 (1993); Clark et al. Gene Therapy 3: 1124-1132 (1996); U.S. Pat. Nos. 5,786,211; 5,871,982; and 6,258,595. The foregoing documents are hereby incorporated by reference in their entirety herein, with particular emphasis on those sections of the documents relating to rAAV production.

Also disclosed herein are packaging cells that produce infectious rAAV. In one embodiment packaging cells may be stably transformed cancer cells such as HeLa cells, 293 cells and PerC.6 cells (a cognate 293 line). In another embodiment, packaging cells are cells that are not transformed cancer cells, such as low passage 293 cells (human fetal kidney cells transformed with El of adenovirus), MRC-5 cells (human fetal fibroblasts), WI-38 cells (human fetal fibroblasts), Vero cells (monkey kidney cells) and FRhL-2 cells (rhesus fetal lung cells).

Recombinant AAV (i.e., infectious encapsidated rAAV particles) of the invention comprise a rAAV genome. In exemplary embodiments, the genomes of both rAAV lack AAV rep and cap DNA, that is, there is no AAV rep or cap DNA between the ITRs of the genomes. Examples of rAAV that may be constructed to comprise the nucleic acid molecules of the invention are set out in International Patent Application No. PCT/US2012/047999 (WO 2013/016352) incorporated by reference herein in its entirety.

The rAAV may be purified by methods standard in the art such as by column chromatography or cesium chloride gradients. Methods for purifying rAAV vectors from helper virus are known in the art and include methods disclosed in, for example, Clark et al., Hum. Gene Ther., 10(6): 1031-1039 (1999); Schenpp and Clark, Methods Mol. Med., 69 427-443 (2002); U.S. Pat. No. 6,566,118 and WO 98/09657.

In another embodiment, the invention contemplates compositions comprising rAAV of the present invention. Compositions of the invention comprise rAAV and a pharmaceutically acceptable carrier. The compositions may also comprise other ingredients such as diluents and adjuvants. Acceptable carriers, diluents and adjuvants are nontoxic to recipients and are preferably inert at the dosages and concentrations employed, and include buffers and surfactants such as pluronics.

Titers of rAAV to be administered in methods of the invention will vary depending, for example, on the particular rAAV, the mode of administration, the treatment goal, the individual, and the cell type(s) being targeted, and may be determined by methods standard in the art. Titers of rAAV may range from about 1.times.10.sup.6, about 1.times.10.sup.7, about 1.times.10.sup.8, about 1.times.10.sup.9, about 1.times.10.sup.10, about 1.times.10.sup.11, about 1.times.10.sup.12, about 1.times.10.sup.13 to about 1.times.10.sup.14 or more DNase resistant particles (DRP) per ml. Dosages may also be expressed in units of viral genomes (vg).

In some aspects, an isolated nucleic acid as described herein comprises a region (e.g., a first region) comprising a first adeno-associated virus (AAV) inverted terminal repeat (ITR), or a variant thereof and a second region comprising a transgene encoding PGM1. The isolated nucleic acid (e.g., the recombinant AAV vector) can be packaged into a capsid protein and administered to a subject and/or delivered to a selected target cell. The transgene can also comprise a region encoding, for example, a protein and/or an expression control sequence (e.g., a poly-A tail).

Also disclosed herein are vectors comprising a single, cis-acting wild-type ITR. In some aspects, the ITR can be a 5′ ITR. In some aspects, the ITR can be a 3′ ITR. ITR sequences are about 145 bp in length. In some aspects, the entire sequences encoding the ITR(s) can be used in the molecule, although some degree of minor modification of these sequences is permissible. In some aspects, an ITR can be mutated at its terminal resolution site (TR), which inhibits replication at the vector terminus where the TR has been mutated and results in the formation of a self-complementary AAV. In some aspects, a “cis-acting” plasmid containing the transgene, in which the selected transgene sequence and associated regulatory elements can be flanked by the 5′ AAV ITR sequence and a 3′ hairpin-forming RNA sequence, can be used. AAV ITR sequences can be obtained from any known AAV, including presently identified mammalian AAV types. In some aspects, an ITR sequence can be an AAV2, AAV3, AAV4, AAV5, AAV6, AAV7, AAV8, AAVrh8, AAV9, AAV10, and/or AAVrh10 ITR sequence. In some aspects, the AAV ITR sequences can be AAV9.

In some aspects, the recombinant AAV genome containing the transgene contains the elements as follows: AAV9ITR-CAG Promoter-PGM1 coding sequence-polyA signal sequence-AAV9ITR sequence. In some aspects, the construct further comprises an intron and or a WPRE sequence. In some aspects, the construct contains AAV9 ITR sequence-CAG promoter-Intron Sequence-PGM1 codon optimized coding sequence-WPRE sequence-bovine growth hormone polyA signal Sequence-AAV9 ITR sequence. In some aspects, the nucleotide sequence of the construct can be SEQ ID NO: 8.

In some aspects, the rAAV can be an AAV9 serotype. Other serotypes with tropism for cardiac cells may also be used. In some aspects, the rAAV can have a capsid having the amino acid sequence of the AAV9 capsid, or that is 99%, 98%, 95%, 90% or 85% identical to the AAV9 capsid. In some aspects, the AAV9 capsid has the amino acid sequence as follows:

(SEQ ID NO: 10)
MAADGYLPDW LEDNLSEGIR EWWALKPGAP QPKANQQHQD
NARGLVLPGY KYLGPGNGLD KGEPVNAADA AALEHDKAYD
QQLKAGDNPY LKYNHADAEF QERLKEDTSF GGNLGRAVFQ
AKKRLLEPLG LVEEAAKTAP GKKRPVEQSP QEPDSSAGIG
KSGAQPAKKR LNFGQTGDTE SVPDPQPIGE PPAAPSGVGS
LTMASGGGAP VADNNEGADG VGSSSGNWHC DSQWLGDRVI
TTSTRTWALP TYNNHLYKQI SNSTSGGSSN DNAYFGYSTP
WGYFDFNRFH CHFSPRDWQR LINNNWGFRP KRLNFKLFNI
QVKEVTDNNG VKTIANNLTS TVQVFTDSDY QLPYVLGSAH
EGCLPPFPAD VFMIPQYGYL TLNDGSQAVG RSSFYCLEYF
PSQMLRTGNN FQFSYEFENV PFHSSYAHSQ SLDRLMNPLI
DQYLYYLSKT INGSGQNQQT LKFSVAGPSN MAVQGRNYIP
GPSYRQQRVS TTVTQNNNSE FAWPGASSWA LNGRNSLMNP
GPAMASHKEG EDRFFPLSGS LIFGKQGTGR DNVDADKVMI
TNEEEIKTTN PVATESYGQV ATNHQSAQAQ AQTGWVQNQG
ILPGMVWQDR DVYLQGPIWA KIPHTDGNFH PSPLMGGFGM
KHPPPQILIK NTPVPADPPT AFNKDKLNSF ITQYSTGQVS
VEIEWELQKE NSKRWNPEIQ YTSNYYKSNN VEFAVNTEGV
YSEPRPIGTR YLTRNL.

In some aspects, the isolated nucleic acids and/or rAAVs described herein can be modified and/or selected to enhance the targeting of the isolated nucleic acids and/or rAAVs to a target tissue (e.g., CNS). Non-limiting methods of modifications and/or selections include AAV capsid serotypes (e.g., AAV9), tissue-specific promoters, and/or targeting peptides. In some aspects, the isolated nucleic acids and rAAVs disclosed herein can comprise AAV capsid serotypes with enhanced targeting to cardiac tissues (e.g., AAV9). In some aspects, the isolated nucleic acids and rAAVs described herein can comprise tissue-specific promoters. In some aspects, the isolated nucleic acids and rAAVs described herein can comprise AAV capsid serotypes with enhanced targeting to cardiac tissues and tissue-specific promoters. While AAV9 targets cardiac tissue, the rAAV9 vectors can also transduce other non-cardiac tissues and, thus, the transgenes, under the control of a promoter such as the CAG promoter can be expressed both in cardiac myocytes and other tissues outside the heart. In some aspects, direct delivery of the constructs disclosed herein can target cardiac tissue resulting in cardiac expression of PGM1 but also lead to PGM1 expression in peripheral tissues including but not limited to liver.

In some aspects, the disclosure provides isolated AAVs. As used herein with respect to AAVs, the term “isolated” refers to an AAV that has been artificially obtained or produced. Isolated AAVs can be produced using recombinant methods. Such AAVs are referred to herein as “recombinant AAVs”. Recombinant AAVs (rAAVs) preferably have tissue-specific targeting capabilities, such that a transgene of the rAAV can be delivered specifically to one or more predetermined tissue(s). The AAV capsid can be an important element in determining these tissue-specific targeting capabilities. Thus, an rAAV having a capsid appropriate for the tissue being targeted can be selected. In some aspects, the rAAV comprises an AAV1, AAV2, AAV3, AAV4, AAV5, AAV6, AAV7, AAV8, AAVrh8, AAV9, AAV10, AAVrh10, or AAV.PHPB capsid protein, or a protein having substantial homology thereto. In some aspects, the rAAV comprises an AAV9 capsid protein. In some aspects, the rAAV comprises an AAVPHP.B capsid protein.

In some aspects, the rAAVs described herein can be pseudotyped rAAVs. Pseudotyping is the process of producing viruses or viral vectors in combination with foreign viral envelope proteins. The result is a pseudotyped virus particle. With this method, the foreign viral envelope proteins can be used to alter host tropism or an increased/decreased stability of the virus particles. In some aspects, a pseudotyped rAAV comprises nucleic acids from two or more different AAVs, wherein the nucleic acid from one AAV encodes a capsid protein and the nucleic acid of at least one other AAV encodes other viral proteins and/or the viral genome. In some aspects, a pseudotyped rAAV refers to an AAV comprising an inverted terminal repeats (ITRs) of one AAV serotype and a capsid protein of a different AAV serotype. For example, a pseudotyped AAV vector containing the ITRs of serotype X encapsidated with the proteins of Y can be designated as AAVX/Y (e.g., AAV2/1 has the ITRs of AAV2 and the capsid of AAV1). In some aspects, pseudotyped rAAVs can be useful for combining the tissue-specific targeting capabilities of a capsid protein from one AAV serotype with the viral DNA from another AAV serotype, thereby allowing targeted delivery of a transgene to a target tissue.

Methods for obtaining recombinant AAVs having a desired capsid protein are well known in the art. (See, for example, US Patent Application Publication Number US 2003/0138772, the contents of which are incorporated herein by reference in their entirety). Typically the methods involve culturing a host cell which contains a nucleic acid sequence encoding an AAV capsid protein or fragment thereof; a functional rep gene; a recombinant AAV vector composed of, AAV inverted terminal repeats (ITRs) and a transgene; and sufficient helper functions to permit packaging of the recombinant AAV vector into the AAV capsid proteins. Typically, capsid proteins are structural proteins encoded by the cap gene of an AAV. In some aspects, AAVs comprise three capsid proteins, virion proteins 1 to 3 (named VP1, VP2 and VP3), which are transcribed from a single cap gene via alternative splicing. In some aspects, the molecular weights of VP1, VP2 and VP3 are respectively about 87 kDa, about 72 kDa and about 62 kDa. In some aspects, upon translation, capsid proteins form a spherical 60-mer protein shell around the viral genome. In some aspects, capsid proteins protect a viral genome, deliver a genome and/or interact with a host cell. In some aspects, capsid proteins deliver the viral genome to a host in a tissue specific manner.

In some aspects, the AAV capsid protein can be an AAV serotype selected from the group consisting of AAV3, AAV4, AAV5, AAV6, AAV8, AAVrh8 AAV9, AAV10 and AAVrh10. In some aspects, the AAV capsid protein can be an AAVrh8, AAVrh10, or AAV.PHPB serotype. In some aspects, the AAV capsid protein can be an AAVrh8 serotype. In some aspects, the AAV capsid protein can be an AAV9 serotype. In some aspects, the AAV capsid protein can be an AAV.PHPB serotype.

In some aspects, components to be cultured in the host cell to package a rAAV vector in an AAV capsid can be provided to the host cell in trans. Alternatively, any one or more of the required components (e.g., recombinant AAV vector, rep sequences, cap sequences, and/or helper functions) can be provided by a stable host cell which has been engineered to contain one or more of the required components using methods known to those of skill in the art.

In some aspects, such a stable host cell can contain the required component(s) under the control of an inducible promoter. However, the required component(s) can be under the control of a constitutive promoter. Examples of suitable inducible and constitutive promoters are provided herein, in the discussion of regulatory elements suitable for use with the transgene. In some aspects, a selected stable host cell can contain selected component(s) under the control of a constitutive promoter and other selected component(s) under the control of one or more inducible promoters. For example, a stable host cell may be generated which is derived from 293 cells (which contain El helper functions under the control of a constitutive promoter), but which contain the rep and/or cap proteins under the control of inducible promoters. Still other stable host cells may be generated by one of skill in the art.

The recombinant AAV vector, rep sequences, cap sequences, and helper functions useful for producing the rAAV described herein can be delivered to the packaging host cell using any appropriate genetic element (vector). The selected genetic element can be delivered by any suitable method, including those described herein. The methods used to construct any of compositions disclosed herein are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al, J. Virol., 70:520-532 (1993) and U.S. Pat. No. 5,478,745.

In some aspects, recombinant AAVs can be produced using the triple transfection method (described in detail in U.S. Pat. No. 6,001,650). Typically, the recombinant AAVs can be produced by transfecting a host cell with a recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. In some aspects, the AAV helper function vector can support efficient AAV vector production without generating any detectable wild-type AAV virions (i.e., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

Cells. Disclosed herein are transfected host cells. The term “transfection” is used to refer to the uptake of foreign DNA by a cell, and a cell has been “transfected” when exogenous DNA has been introduced through the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

As used herein, the term “host cell” refers to any cell that harbors, or is capable of harboring, a substance of interest. Often a host cell can be a mammalian cell (e.g., a non-human primate, rodent, or human cell). In some aspects, the host cell can be a mammalian cell, a yeast cell, a bacterial cell, an insect cell, a plant cell, or a fungal cell. A host cell can be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein can refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

Provided herein are host cells for production of rAAV, particularly rAAV9 particles, containing a genome comprising a transgene encoding PGM1 (including the nucleotide sequence of SEQ ID NO: 1) operably linked to regulatory elements that promote expression of the PGM1 transgene in vivo. For example, operably linked to a CAG promoter and a polyA signal sequence. The gene expression cassette can have the nucleotide sequence of SEQ ID NO: 9 and can include flanking ITR sequences, for example, the entire construct with the flanking ITR sequences can have the nucleotide sequence of SEQ ID NO: 8.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the term “recombinant cell” refers to a cell into which an exogenous DNA segment, such as DNA segment that leads to the transcription of a biologically-active polypeptide or production of a biologically active nucleic acid such as an RNA, has been introduced.

TABLE 1
Sequences

| Sequence | Name | SEQ ID NO: |
|---|---|---|
| ATGGTGAAG ATCGTGACAG TTAAGACCCA | hPGM1 | 1 |
| GGCGTACCAG GACCAGAAGC CGGGCACGAG | | |
| CGGGCTGCGGAAGCGGGTGA AGGTGTTCCA | | |
| GAGCAGCGCC AACTACGCGG AGAACTTCAT | | |
| CCAGAGTATC ATCTCCACCG TGGAGCCGGC | | |
| GCAGCGGCAG GAGGCCACGC TGGTGGTGGG | | |
| CGGGGACGGC CGGTTCTACA TGAAGGAGGC | | |
| CATCCAGCTC ATCGCTCGCA TCGCTGCCGC | | |
| CAACGGGATC GGTCGCTTGG TTATCGGACA | | |
| GAATGGAATC CTCTCCACCC CTGCTGTATC | | |
| CTGCATCATT AGAAAAATCA AAGCCATTGG | | |
| TGGGATCATT CTGACAGCCA GTCACAACCC | | |
| AGGGGGCCCC AATGGAGATT TTGGAATCAA | | |
| ATTCAATATT TCTAATGGAG GTCCTGCTCC | | |
| AGAAGCAATA ACTGATAAAA TTTTCCAAAT | | |
| CAGCAAGACA ATTGAAGAAT ATGCAGTTTG | | |
| CCCTGACCTG AAAGTAGACC TTGGTGTTCT | | |
| GGGAAAGCAG CAGTTTGACT TGGAAAATAA | | |
| GTTCAAACCC TTCACAGTGG AAATTGTGGA | | |
| TTCGGTAGAA GCTTATGCTA CAATGCTGAG | | |
| AAGCATCTTT GATTTCAGTG CACTGAAAGA | | |
| ACTACTTTCT GGGCCAAACC GACTGAAGAT | | |
| CCGTATTGAT GCTATGCATG GAGTTGTGGG | | |
| ACCGTATGTA AAGAAGATCC TCTGTGAAGA | | |
| ACTCGGTGCC CCTGCGAACT CGGCAGTTAA | | |
| CTGCGTTCCT CTGGAGGACT TTGGAGGCCA | | |
| CCACCCTGAC CCCAACCTCA CCTATGCAGC | | |
| TGACCTGGTG GAGACCATGA AGTCAGGAGA | | |
| GCATGATTTT GGGGCTGCCT TTGATGGAGA | | |
| TGGGGATCGA AACATGATTC TGGGCAAGCA | | |
| TGGGTTCTTT GTGAACCCTT CAGACTCTGT | | |
| GGCTGTCATT GCTGCCAACA TCTTCAGCAT | | |
| TCCGTATTTC CAGCAGACTG GGGTCCGCGG | | |
| CTTTGCACGG AGCATGCCCA CGAGTGGTGC | | |
| TCTGGACCGG GTGGCTAGTG CTACAAAGAT | | |
| TGCTTTGTAT GAGACCCCAA CTGGCTGGAA | | |
| GTTTTTTGGG AATTTGATGG ACGCGAGCAA | | |
| ACTGTCCCTT TGTGGGGAGG AGAGCTTCGG | | |
| GACCGGTTCT GACCACATCC GTGAGAAAGA | | |
| TGGACTGTGG GCTGTCCTTG CCTGGCTCTC | | |
| CATCCTAGCC ACCCGCAAGC AGAGTGTGGA | | |
| GGACATTCTC AAAGATCATT GGCAAAAGTA | | |
| TGGCCGGAAT TTCTTCACCA GGTATGATTA | | |
| CGAGGAGGTG GAAGCTGAGG GCGCAAACAA | | |
| AATGATGAAG GACTTGGAGG CCCTGATGTT | | |
| TGATCGCTCC TTTGTGGGGA AGCAGTTCTC | | |
| AGCAAATGAC AAAGTTTACA CTGTGGAGAA | | |
| GGCCGATAAC TTTGAATACA GCGACCCAGT | | |
| GGATGGAAGC ATTTCAAGAA ATCAGGGCTT | | |
| GCGCCTCATT TTCACAGATG GTTCTCGAAT | | |
| CGTCTTCCGA CTGAGCGGCA CTGGGAGTGC | | |
| CGGGGCCACC ATTCGGCTGT ACATCGATAG | | |
| CTATGAGAAG GACGTTGCCA AGATTAACCA | | |
| GGACCCCCAG GTCATGTTGG CCCCCCTTAT | | |
| TTCCATTGCT CTGAAAGTGT CCCAGCTGCA | | |
| GGAGAGGACG GGACGCACTG CACCCACTGT | | |
| CATCACCTAA | | |
| CT CGACATTGAT TATTGACTAG TTATTAATAG | CAG | 2 |
| TAATCAATTA CGGGGTCATT AGTTCATAGC | promoter | |
| CCATATATGG AGTTCCGCGT TACATAACTT | | |
| ACGGTAAATG GCCCGCCTGG CTGACCGCCC | | |
| AACGACCCCC GCCCATTGAC GTCAATAATG | | |
| ACGTATGTTC CCATAGTAAC GCCAATAGGG | | |
| ACTTTCCATT GACGTCAATG GGTGGAGTAT | | |
| TTACGGTAAA CTGCCCACTT GGCAGTACAT | | |
| CAAGTGTATC ATATGCCAAG TACGCCCCCT | | |
| ATTGACGTCA ATGACGGTAA ATGGCCCGCC | | |
| TGGCATTATG CCCAGTACAT GACCTTATGG | | |
| GACTTTCCTA CTTGGCAGTA CATCTACGTA | | |
| TTAGTCATCG CTATTACCAT GGTCGAGGTG | | |
| AGCCCCACGT TCTGCTTCAC TCTCCCCATC | | |
| TCCCCCCCCT CCCCACCCCC AATTTTGTAT | | |
| TTATTTATTT TTTAATTATT TTGTGCAGCG | | |
| ATGGGGGCGG GGGGGGGGGG GGGGCGCGCG | | |
| CCAGGCGGGG CGGGGCGGGG CGAGGGGCGG | | |
| GGCGGGGCGA GGCGGAGAGG TGCGGCGGCA | | |
| GCCAATCAGA GCGGCGCGCT CCGAAAGTTT | | |
| CCTTTTATGG CGAGGCGGCG GCGGCGGCGG | | |
| CCCTATAAAA AGCGAAGCGC GCGGCGGGCG | | |
| GGAGTCGCTG CGCGCTGCCT TCGCCCCGTG | | |
| CCCCGCTCCG CCGCCGCCTC GCGCCGCCCG | | |
| CCCCGGCTCT GACTGACCGC GTTACTCCCA | | |
| CAGGTGAGCG GGCGGGACGG CCCTTCTCCT | | |
| CCGGGCTGTA ATTAGCGCTT GGTTTAATGA | | |
| CGGCTTGTTT CTTTTCTGTG GCTGCGTGAA | | |
| AGCCTTGAGG GGCTCCGGGA GGGCCCTTTG | | |
| TGCGGGGGGA GCGGCTCGGG GGGTGCGTGC | | |
| GTGTGTGTGT GCGTGGGGAG CGCCGCGTGC | | |
| GGCTCCGCGC TGCCCGGCGG CTGTGAGCGC | | |
| TGCGGGCGCG GCGCGGGGCT TTGTGCGCTC | | |
| CGCAGTGTGC GCGAGGGGAG CGCGGCCGGG | | |
| GGCGGTGCCC CGCGGTGCGG GGGGGGCTGC | | |
| GAGGGGAACA AAGGCTGCGT GCGGGGTGTG | | |
| TGCGTGGGGG GGTGAGCAGG GGGTGTGGGC | | |
| GCGTCGGTCG GGCTGCAACC CCCCCTGCAC | | |
| CCCCCTCCCC GAGTTGCTGA GCACGGCCCG | | |
| GCTTCGGGTG CGGGGCTCCG TACGGGGCGT | | |
| GGCGCGGGGC TCGCCGTGCC GGGCGGGGGG | | |
| TGGCGGCAGG TGGGGGTGCC GGGCGGGGCG | | |
| GGGCCGCCTC GGGCCGGGGA GGGCTCGGGG | | |
| GAGGGGCGCG GCGGCCCCCG GAGCGCCGGC | | |
| GGCTGTCGAG GCGCGGCGAG CCGCAGCCAT | | |
| TGCCTTTTAT GGTAATCGTG CGAGAGGGCG | | |
| CAGGGACTTC CTTTGTCCCA AATCTGTGCG | | |
| GAGCCGAAAT CTGGGAGGCG CCGCCGCACC | | |
| CCCTCTAGCG GGCGCGGGGC GAAGCGGTGC | | |
| GGCGCCGGCA GGAAGGAAAT GGGGGGGGAG | | |
| GGCCTTCGTG CGTCGCCGCG CCGCCGTCCC | | |
| CTTCTCCCTC TCCAGCCTCG GGGCTGTCCG | | |
| CGGGGGGACG GCTGCCTTCG GGGGGGACGG | | |
| GGCAGGGCGG GGTTCGGCTT CTGGCGTGTG | | |
| ACCGGCGGCT CTAGAGCCTC TGCTAACCAT | | |
| GTTCATGCCT TCTTCTTTTT CCTACAGCTC | | |
| CTGGGCAACG TGCTGGTTAT TGTGCTGTCT | | |
| CATCATTTTG GCAAAGAATT G | | |
| CT GTGCCTTCTA GTTGCCAGCC ATCTGTTGTT | Bgh pA | 3 |
| TGCCCCTCCC CCGTGCCTTC CTTGACCCTG | | |
| GAAGGTGCCA CTCCCACTGT CCTTTCCTAA | | |
| TAAAATGAGG AAATTGCATC GCATTGTCTG | | |
| AGTAGGTGTC ATTCTATTCT GGGGGGTGGG | | |
| GTGGGGCAGG ACAGCAAGGG GGAGGATTGG | | |
| GAAGAGAATA GCAGGCATGC TGGGGA | | |
| GCCAC C | Kozak | 4 |
| CCTGCAGGCA GCTGCGCGCT CGCTCGCTCA | 5′ ITR | 5 |
| CTGAGGCCGC CCGGGCAAAG CCCGGGCGTC | | |
| GGGCGACCTT TGGTCGCCCG GCCTCAGTGA | | |
| GCGAGCGAGC GCGCAGAGAG GGAGTGGCCA | | |
| ACTCCATCAC TAGGGGTTCC T | | |
| AGGAACC CCTAGTGATG GAGTTGGCCA | 3′ ITR | 6 |
| CTCCCTCTCT GCGCGCTCGC TCGCTCACTG | | |
| AGGCCGGGCG | | |
| ACCAAAGGTC GCCCGACGCC CGGGCTTTGC | | |
| CCGGGCGGCC TCAGTGAGCG AGCGAGCGCG | | |
| CAGCTGCCTG CAGG | | |
| CGATAATCAA CCTCTGGATT ACAAAATTTG | WPRE | 7 |
| TGAAAGATTG ACTGGTATTC TTAACTATGT | | |
| TGCTCCTTTT ACGCTATGTG GATACGCTGC | | |
| TTTAATGCCT TTGTATCATG CTATTGCTTC | | |
| CCGTATGGCT TTCATTTTCT CCTCCTTGTA | | |
| TAAATCCTGG TTGCTGTCTC TTTATGAGGA | | |
| GTTGTGGCCC GTTGTCAGGC AACGTGGCGT | | |
| GGTGTGCACT GTGTTTGCTG ACGCAACCCC | | |
| CACTGGTTGG GGCATTGCCA CCACCTGTCA | | |
| GCTCCTTTCC GGGACTTTCG CTTTCCCCCT | | |
| CCCTATTGCC ACGGCGGAAC TCATCGCCGC | | |
| CTGCCTTGCC CGCTGCTGGA CAGGGGCTCG | | |
| GCTGTTGGGC ACTGACAATT CCGTGGTGTT | | |
| GTCGGGGAAG CTGACGTCCT TTCCATGGCT | | |
| GCTCGCCTGT GTTGCCACCT GGATTCTGCG | | |
| CGGGACGTCC TTCTGCTACG TCCCTTCGGC | | |
| CCTCAATCCA GCGGACCTTC CTTCCCGCGG | | |
| CCTGCTGCCG GCTCTGCGGC CTCTTCCGCG | | |
| TCTTCGCCTT CGCCCTCAGA CGAGTCGGAT | | |
| CTCCCTTTGG GCCGCCTCCC CGCATCGG | | |
| CCTGCAGGCA GCTGCGCGCT CGCTCGCTCA | Nucleic | 8 |
| CTGAGGCCGC CCGGGCAAAG CCCGGGCGTC | acid | |
| GGGCGACCTT TGGTCGCCCG GCCTCAGTGA | construct | |
| GCGAGCGAGC GCGCAGAGAG GGAGTGGCCA | with ITRs | |
| ACTCCATCAC TAGGGGTTCC TTCTAGACAA | | |
| CTTTGTATAG AAAAGTTGCT CGACATTGAT | | |
| TATTGACTAG TTATTAATAG TAATCAATTA | | |
| CGGGGTCATT AGTTCATAGC CCATATATGG | | |
| AGTTCCGCGT TACATAACTT ACGGTAAATG | | |
| GCCCGCCTGG CTGACCGCCC AACGACCCCC | | |
| GCCCATTGAC GTCAATAATG ACGTATGTTC | | |
| CCATAGTAAC GCCAATAGGG ACTTTCCATT | | |
| GACGTCAATG GGTGGAGTAT TTACGGTAAA | | |
| CTGCCCACTT GGCAGTACAT CAAGTGTATC | | |
| ATATGCCAAG TACGCCCCCT ATTGACGTCA | | |
| ATGACGGTAA ATGGCCCGCC TGGCATTATG | | |
| CCCAGTACAT GACCTTATGG GACTTTCCTA | | |
| CTTGGCAGTA CATCTACGTA TTAGTCATCG | | |
| CTATTACCAT GGTCGAGGTG AGCCCCACGT | | |
| TCTGCTTCAC TCTCCCCATC TCCCCCCCCT | | |
| CCCCACCCCC AATTTTGTAT TTATTTATTT | | |
| TTTAATTATT TTGTGCAGCG ATGGGGGCGG | | |
| GGGGGGGGGG GGGGCGCGCG CCAGGCGGGG | | |
| CGGGGCGGGG CGAGGGGCGG GGCGGGGCGA | | |
| GGCGGAGAGG TGCGGCGGCA GCCAATCAGA | | |
| GCGGCGCGCT CCGAAAGTTT CCTTTTATGG | | |
| CGAGGCGGCG GCGGCGGCGG CCCTATAAAA | | |
| AGCGAAGCGC GCGGCGGGCG GGAGTCGCTG | | |
| CGCGCTGCCT TCGCCCCGTG CCCCGCTCCG | | |
| CCGCCGCCTC GCGCCGCCCG CCCCGGCTCT | | |
| GACTGACCGC GTTACTCCCA CAGGTGAGCG | | |
| GGCGGGACGG CCCTTCTCCT CCGGGCTGTA | | |
| ATTAGCGCTT GGTTTAATGA CGGCTTGTTT | | |
| CTTTTCTGTG GCTGCGTGAA AGCCTTGAGG | | |
| GGCTCCGGGA GGGCCCTTTG TGCGGGGGGA | | |
| GCGGCTCGGG GGGTGCGTGC GTGTGTGTGT | | |
| GCGTGGGGAG CGCCGCGTGC GGCTCCGCGC | | |
| TGCCCGGCGG CTGTGAGCGC TGCGGGCGCG | | |
| GCGCGGGGCT TTGTGCGCTC CGCAGTGTGC | | |
| GCGAGGGGAG CGCGGCCGGG GGCGGTGCCC | | |
| CGCGGTGCGG GGGGGGCTGC GAGGGGAACA | | |
| AAGGCTGCGT GCGGGGTGTG TGCGTGGGGG | | |
| GGTGAGCAGG GGGTGTGGGC GCGTCGGTCG | | |
| GGCTGCAACC CCCCCTGCAC CCCCCTCCCC | | |
| GAGTTGCTGA GCACGGCCCG GCTTCGGGTG | | |
| CGGGGCTCCG TACGGGGCGT GGCGCGGGGC | | |
| TCGCCGTGCC GGGCGGGGGG TGGCGGCAGG | | |
| TGGGGGTGCC GGGCGGGGCG GGGCCGCCTC | | |
| GGGCCGGGGA GGGCTCGGGG GAGGGGCGCG | | |
| GCGGCCCCCG GAGCGCCGGC GGCTGTCGAG | | |
| GCGCGGCGAG CCGCAGCCAT TGCCTTTTAT | | |
| GGTAATCGTG CGAGAGGGCG CAGGGACTTC | | |
| CTTTGTCCCA AATCTGTGCG GAGCCGAAAT | | |
| CTGGGAGGCG CCGCCGCACC CCCTCTAGCG | | |
| GGCGCGGGGC GAAGCGGTGC GGCGCCGGCA | | |
| GGAAGGAAAT GGGGGGGGAG GGCCTTCGTG | | |
| CGTCGCCGCG CCGCCGTCCC CTTCTCCCTC | | |
| TCCAGCCTCG GGGCTGTCCG CGGGGGGACG | | |
| GCTGCCTTCG GGGGGGACGG GGCAGGGCGG | | |
| GGTTCGGCTT CTGGCGTGTG ACCGGCGGCT | | |
| CTAGAGCCTC TGCTAACCAT GTTCATGCCT | | |
| TCTTCTTTTT CCTACAGCTC CTGGGCAACG | | |
| TGCTGGTTAT TGTGCTGTCT CATCATTTTG | | |
| GCAAAGAATT GCAAGTTTGT ACAAAAAAGC | | |
| AGGCTGCCAC CATGGTGAAG ATCGTGACAG | | |
| TTAAGACCCA GGCGTACCAG GACCAGAAGC | | |
| CGGGCACGAG CGGGCTGCGG AAGCGGGTGA | | |
| AGGTGTTCCA GAGCAGCGCC AACTACGCGG | | |
| AGAACTTCAT CCAGAGTATC ATCTCCACCG | | |
| TGGAGCCGGC GCAGCGGCAG GAGGCCACGC | | |
| TGGTGGTGGG CGGGGACGGC CGGTTCTACA | | |
| TGAAGGAGGC CATCCAGCTC ATCGCTCGCA | | |
| TCGCTGCCGC CAACGGGATC GGTCGCTTGG | | |
| TTATCGGACA GAATGGAATC CTCTCCACCC | | |
| CTGCTGTATC CTGCATCATT AGAAAAATCA | | |
| AAGCCATTGG TGGGATCATT CTGACAGCCA | | |
| GTCACAACCC AGGGGGCCCC AATGGAGATT | | |
| TTGGAATCAA ATTCAATATT TCTAATGGAG | | |
| GTCCTGCTCC AGAAGCAATA ACTGATAAAA | | |
| TTTTCCAAAT CAGCAAGACA ATTGAAGAAT | | |
| ATGCAGTTTG CCCTGACCTG AAAGTAGACC | | |
| TTGGTGTTCT GGGAAAGCAG CAGTTTGACT | | |
| TGGAAAATAA GTTCAAACCC TTCACAGTGG | | |
| AAATTGTGGA TTCGGTAGAA GCTTATGCTA | | |
| CAATGCTGAG AAGCATCTTT GATTTCAGTG | | |
| CACTGAAAGA ACTACTTTCT GGGCCAAACC | | |
| GACTGAAGAT CCGTATTGAT GCTATGCATG | | |
| GAGTTGTGGG ACCGTATGTA AAGAAGATCC | | |
| TCTGTGAAGA ACTCGGTGCC CCTGCGAACT | | |
| CGGCAGTTAA CTGCGTTCCT CTGGAGGACT | | |
| TTGGAGGCCA CCACCCTGAC CCCAACCTCA | | |
| CCTATGCAGC TGACCTGGTG GAGACCATGA | | |
| AGTCAGGAGA GCATGATTTT GGGGCTGCCT | | |
| TTGATGGAGA TGGGGATCGA AACATGATTC | | |
| TGGGCAAGCA TGGGTTCTTT GTGAACCCTT | | |
| CAGACTCTGT GGCTGTCATT GCTGCCAACA | | |
| TCTTCAGCAT TCCGTATTTC CAGCAGACTG | | |
| GGGTCCGCGG CTTTGCACGG AGCATGCCCA | | |
| CGAGTGGTGC TCTGGACCGG GTGGCTAGTG | | |
| CTACAAAGAT TGCTTTGTAT GAGACCCCAA | | |
| CTGGCTGGAA GTTTTTTGGG AATTTGATGG | | |
| ACGCGAGCAA ACTGTCCCTT TGTGGGGAGG | | |
| AGAGCTTCGG GACCGGTTCT GACCACATCC | | |
| GTGAGAAAGA TGGACTGTGG GCTGTCCTTG | | |
| CCTGGCTCTC CATCCTAGCC ACCCGCAAGC | | |
| AGAGTGTGGA GGACATTCTC AAAGATCATT | | |
| GGCAAAAGTA TGGCCGGAAT TTCTTCACCA | | |
| GGTATGATTA CGAGGAGGTG GAAGCTGAGG | | |
| GCGCAAACAA AATGATGAAG GACTTGGAGG | | |
| CCCTGATGTT TGATCGCTCC TTTGTGGGGA | | |
| AGCAGTTCTC AGCAAATGAC AAAGTTTACA | | |
| CTGTGGAGAA GGCCGATAAC TTTGAATACA | | |
| GCGACCCAGT GGATGGAAGC ATTTCAAGAA | | |
| ATCAGGGCTT GCGCCTCATT TTCACAGATG | | |
| GTTCTCGAAT CGTCTTCCGA CTGAGCGGCA | | |
| CTGGGAGTGC CGGGGCCACC ATTCGGCTGT | | |
| ACATCGATAG CTATGAGAAG GACGTTGCCA | | |
| AGATTAACCA GGACCCCCAG GTCATGTTGG | | |
| CCCCCCTTAT TTCCATTGCT CTGAAAGTGT | | |
| CCCAGCTGCA GGAGAGGACG GGACGCACTG | | |
| CACCCACTGT CATCACCTAA ACCCAGCTTT | | |
| CTTGTACAAA GTGGGAATTC CGATAATCAA | | |
| CCTCTGGATT ACAAAATTTG TGAAAGATTG | | |
| ACTGGTATTC TTAACTATGT TGCTCCTTTT | | |
| ACGCTATGTG GATACGCTGC TTTAATGCCT | | |
| TTGTATCATG CTATTGCTTC CCGTATGGCT | | |
| TTCATTTTCT CCTCCTTGTA TAAATCCTGG | | |
| TTGCTGTCTC TTTATGAGGA GTTGTGGCCC | | |
| GTTGTCAGGC AACGTGGCGT GGTGTGCACT | | |
| GTGTTTGCTG ACGCAACCCC CACTGGTTGG | | |
| GGCATTGCCA CCACCTGTCA GCTCCTTTCC | | |
| GGGACTTTCG CTTTCCCCCT CCCTATTGCC | | |
| ACGGCGGAAC TCATCGCCGC CTGCCTTGCC | | |
| CGCTGCTGGA CAGGGGCTCG GCTGTTGGGC | | |
| ACTGACAATT CCGTGGTGTT GTCGGGGAAG | | |
| CTGACGTCCT TTCCATGGCT GCTCGCCTGT | | |
| GTTGCCACCT GGATTCTGCG CGGGACGTCC | | |
| TTCTGCTACG TCCCTTCGGC CCTCAATCCA | | |
| GCGGACCTTC CTTCCCGCGG CCTGCTGCCG | | |
| GCTCTGCGGC CTCTTCCGCG TCTTCGCCTT | | |
| CGCCCTCAGA CGAGTCGGAT CTCCCTTTGG | | |
| GCCGCCTCCC CGCATCGGGA ATTCCTAGAG | | |
| CTCGCTGATC AGCCTCGACT GTGCCTTCTA | | |
| GTTGCCAGCC ATCTGTTGTT TGCCCCTCCC | | |
| CCGTGCCTTC CTTGACCCTG GAAGGTGCCA | | |
| CTCCCACTGT CCTTTCCTAA TAAAATGAGG | | |
| AAATTGCATC GCATTGTCTG AGTAGGTGTC | | |
| ATTCTATTCT GGGGGGTGGG GTGGGGCAGG | | |
| ACAGCAAGGG GGAGGATTGG GAAGAGAATA | | |
| GCAGGCATGC TGGGGAGGGC CGCAGGAACC | | |
| CCTAGTGATG GAGTTGGCCA CTCCCTCTCT | | |
| GCGCGCTCGC TCGCTCACTG AGGCCGGGCG | | |
| ACCAAAGGTC GCCCGACGCC CGGGCTTTGC | | |
| CCGGGCGGCC TCAGTGAGCG AGCGAGCGCG | | |
| CAGCTGCCTG CAGG | | |
| TCTAGACAA CTTTGTATAG AAAAGTTGCT | Nucleic | 9 |
| CGACATTGAT TATTGACTAG TTATTAATAG | acid | |
| TAATCAATTA CGGGGTCATT AGTTCATAGC | construct | |
| CCATATATGG AGTTCCGCGT TACATAACTT | without | |
| ACGGTAAATG GCCCGCCTGG CTGACCGCCC | ITR | |
| AACGACCCCC GCCCATTGAC GTCAATAATG | | |
| ACGTATGTTC CCATAGTAAC GCCAATAGGG | | |
| ACTTTCCATT GACGTCAATG GGTGGAGTAT | | |
| TTACGGTAAA CTGCCCACTT GGCAGTACAT | | |
| CAAGTGTATC ATATGCCAAG TACGCCCCCT | | |
| ATTGACGTCA ATGACGGTAA ATGGCCCGCC | | |
| TGGCATTATG CCCAGTACAT GACCTTATGG | | |
| GACTTTCCTA CTTGGCAGTA CATCTACGTA | | |
| TTAGTCATCG CTATTACCAT GGTCGAGGTG | | |
| AGCCCCACGT TCTGCTTCAC TCTCCCCATC | | |
| TCCCCCCCCT CCCCACCCCC AATTTTGTAT | | |
| TTATTTATTT TTTAATTATT TTGTGCAGCG | | |
| ATGGGGGCGG GGGGGGGGGG GGGGCGCGCG | | |
| CCAGGCGGGG CGGGGCGGGG CGAGGGGCGG | | |
| GGCGGGGCGA GGCGGAGAGG TGCGGCGGCA | | |
| GCCAATCAGA GCGGCGCGCT CCGAAAGTTT | | |
| CCTTTTATGG CGAGGCGGCG GCGGCGGCGG | | |
| CCCTATAAAA AGCGAAGCGC GCGGCGGGCG | | |
| GGAGTCGCTG CGCGCTGCCT TCGCCCCGTG | | |
| CCCCGCTCCG CCGCCGCCTC GCGCCGCCCG | | |
| CCCCGGCTCT GACTGACCGC GTTACTCCCA | | |
| CAGGTGAGCG GGCGGGACGG CCCTTCTCCT | | |
| CCGGGCTGTA ATTAGCGCTT GGTTTAATGA | | |
| CGGCTTGTTT CTTTTCTGTG GCTGCGTGAA | | |
| AGCCTTGAGG GGCTCCGGGA GGGCCCTTTG | | |
| TGCGGGGGGA GCGGCTCGGG GGGTGCGTGC | | |
| GTGTGTGTGT GCGTGGGGAG CGCCGCGTGC | | |
| GGCTCCGCGC TGCCCGGCGG CTGTGAGCGC | | |
| TGCGGGCGCG GCGCGGGGCT TTGTGCGCTC | | |
| CGCAGTGTGC GCGAGGGGAG CGCGGCCGGG | | |
| GGCGGTGCCC CGCGGTGCGG GGGGGGCTGC | | |
| GAGGGGAACA AAGGCTGCGT GCGGGGTGTG | | |
| TGCGTGGGGG GGTGAGCAGG GGGTGTGGGC | | |
| GCGTCGGTCG GGCTGCAACC CCCCCTGCAC | | |
| CCCCCTCCCC GAGTTGCTGA GCACGGCCCG | | |
| GCTTCGGGTG CGGGGCTCCG TACGGGGCGT | | |
| GGCGCGGGGC TCGCCGTGCC GGGCGGGGGG | | |
| TGGCGGCAGG TGGGGGTGCC GGGCGGGGCG | | |
| GGGCCGCCTC GGGCCGGGGA GGGCTCGGGG | | |
| GAGGGGCGCG GCGGCCCCCG GAGCGCCGGC | | |
| GGCTGTCGAG GCGCGGCGAG CCGCAGCCAT | | |
| TGCCTTTTAT GGTAATCGTG CGAGAGGGCG | | |
| CAGGGACTTC CTTTGTCCCA AATCTGTGCG | | |
| GAGCCGAAAT CTGGGAGGCG CCGCCGCACC | | |
| CCCTCTAGCG GGCGCGGGGC GAAGCGGTGC | | |
| GGCGCCGGCA GGAAGGAAAT GGGCGGGGAG | | |
| GGCCTTCGTG CGTCGCCGCG CCGCCGTCCC | | |
| CTTCTCCCTC TCCAGCCTCG GGGCTGTCCG | | |
| CGGGGGGACG GCTGCCTTCG GGGGGGACGG | | |
| GGCAGGGCGG GGTTCGGCTT CTGGCGTGTG | | |
| ACCGGCGGCT CTAGAGCCTC TGCTAACCAT | | |
| GTTCATGCCT TCTTCTTTTT CCTACAGCTC | | |
| CTGGGCAACG TGCTGGTTAT TGTGCTGTCT | | |
| CATCATTTTG GCAAAGAATT GCAAGTTTGT | | |
| ACAAAAAAGC AGGCTGCCAC CATGGTGAAG | | |
| ATCGTGACAG TTAAGACCCA GGCGTACCAG | | |
| GACCAGAAGC CGGGCACGAG CGGGCTGCGG | | |
| AAGCGGGTGA AGGTGTTCCA GAGCAGCGCC | | |
| AACTACGCGG AGAACTTCAT CCAGAGTATC | | |
| ATCTCCACCG TGGAGCCGGC GCAGCGGCAG | | |
| GAGGCCACGC TGGTGGTGGG CGGGGACGGC | | |
| CGGTTCTACA TGAAGGAGGC CATCCAGCTC | | |
| ATCGCTCGCA TCGCTGCCGC CAACGGGATC | | |
| GGTCGCTTGG TTATCGGACA GAATGGAATC | | |
| CTCTCCACCC CTGCTGTATC CTGCATCATT | | |
| AGAAAAATCA AAGCCATTGG TGGGATCATT | | |
| CTGACAGCCA GTCACAACCC AGGGGGCCCC | | |
| AATGGAGATT TTGGAATCAA ATTCAATATT | | |
| TCTAATGGAG GTCCTGCTCC AGAAGCAATA | | |
| ACTGATAAAA TTTTCCAAAT CAGCAAGACA | | |
| ATTGAAGAAT ATGCAGTTTG CCCTGACCTG | | |
| AAAGTAGACC TTGGTGTTCT GGGAAAGCAG | | |
| CAGTTTGACT TGGAAAATAA GTTCAAACCC | | |
| TTCACAGTGG AAATTGTGGA TTCGGTAGAA | | |
| GCTTATGCTA CAATGCTGAG AAGCATCTTT | | |
| GATTTCAGTG CACTGAAAGA ACTACTTTCT | | |
| GGGCCAAACC GACTGAAGAT CCGTATTGAT | | |
| GCTATGCATG GAGTTGTGGG ACCGTATGTA | | |
| AAGAAGATCC TCTGTGAAGA ACTCGGTGCC | | |
| CCTGCGAACT CGGCAGTTAA CTGCGTTCCT | | |
| CTGGAGGACT TTGGAGGCCA CCACCCTGAC | | |
| CCCAACCTCA CCTATGCAGC TGACCTGGTG | | |
| GAGACCATGA AGTCAGGAGA GCATGATTTT | | |
| GGGGCTGCCT TTGATGGAGA TGGGGATCGA | | |
| AACATGATTC TGGGCAAGCA TGGGTTCTTT | | |
| GTGAACCCTT CAGACTCTGT GGCTGTCATT | | |
| GCTGCCAACA TCTTCAGCAT TCCGTATTTC | | |
| CAGCAGACTG GGGTCCGCGG CTTTGCACGG | | |
| AGCATGCCCA CGAGTGGTGC TCTGGACCGG | | |
| GTGGCTAGTG CTACAAAGAT TGCTTTGTAT | | |
| GAGACCCCAA CTGGCTGGAA GTTTTTTGGG | | |
| AATTTGATGG ACGCGAGCAA ACTGTCCCTT | | |
| TGTGGGGAGG AGAGCTTCGG GACCGGTTCT | | |
| GACCACATCC GTGAGAAAGA TGGACTGTGG | | |
| GCTGTCCTTG CCTGGCTCTC CATCCTAGCC | | |
| ACCCGCAAGC AGAGTGTGGA GGACATTCTC | | |
| AAAGATCATT GGCAAAAGTA TGGCCGGAAT | | |
| TTCTTCACCA GGTATGATTA CGAGGAGGTG | | |
| GAAGCTGAGG GCGCAAACAA AATGATGAAG | | |
| GACTTGGAGG CCCTGATGTT TGATCGCTCC | | |
| TTTGTGGGGA AGCAGTTCTC AGCAAATGAC | | |
| AAAGTTTACA CTGTGGAGAA GGCCGATAAC | | |
| TTTGAATACA GCGACCCAGT GGATGGAAGC | | |
| ATTTCAAGAA ATCAGGGCTT GCGCCTCATT | | |
| TTCACAGATG GTTCTCGAAT CGTCTTCCGA | | |
| CTGAGCGGCA CTGGGAGTGC CGGGGCCACC | | |
| ATTCGGCTGT ACATCGATAG CTATGAGAAG | | |
| GACGTTGCCA AGATTAACCA GGACCCCCAG | | |
| GTCATGTTGG CCCCCCTTAT TTCCATTGCT | | |
| CTGAAAGTGT CCCAGCTGCA GGAGAGGACG | | |
| GGACGCACTG CACCCACTGT CATCACCTAA | | |
| ACCCAGCTTT CTTGTACAAA GTGGGAATTC | | |
| CGATAATCAA CCTCTGGATT ACAAAATTTG | | |
| TGAAAGATTG ACTGGTATTC TTAACTATGT | | |
| TGCTCCTTTT ACGCTATGTG GATACGCTGC | | |
| TTTAATGCCT TTGTATCATG CTATTGCTTC | | |
| CCGTATGGCT TTCATTTTCT CCTCCTTGTA | | |
| TAAATCCTGG TTGCTGTCTC TTTATGAGGA | | |
| GTTGTGGCCC GTTGTCAGGC AACGTGGCGT | | |
| GGTGTGCACT GTGTTTGCTG ACGCAACCCC | | |
| CACTGGTTGG GGCATTGCCA CCACCTGTCA | | |
| GCTCCTTTCC GGGACTTTCG CTTTCCCCCT | | |
| CCCTATTGCC ACGGCGGAAC TCATCGCCGC | | |
| CTGCCTTGCC CGCTGCTGGA CAGGGGCTCG | | |
| GCTGTTGGGC ACTGACAATT CCGTGGTGTT | | |
| GTCGGGGAAG CTGACGTCCT TTCCATGGCT | | |
| GCTCGCCTGT GTTGCCACCT GGATTCTGCG | | |
| CGGGACGTCC TTCTGCTACG TCCCTTCGGC | | |
| CCTCAATCCA GCGGACCTTC CTTCCCGCGG | | |
| CCTGCTGCCG GCTCTGCGGC CTCTTCCGCG | | |
| TCTTCGCCTT CGCCCTCAGA CGAGTCGGAT | | |
| CTCCCTTTGG GCCGCCTCCC CGCATCGGGA | | |
| ATTCCTAGAG CTCGCTGATC AGCCTCGACT | | |
| GTGCCTTCTA GTTGCCAGCC ATCTGTTGTT | | |
| TGCCCCTCCC CCGTGCCTTC CTTGACCCTG | | |
| GAAGGTGCCA CTCCCACTGT CCTTTCCTAA | | |
| TAAAATGAGG AAATTGCATC GCATTGTCTG | | |
| AGTAGGTGTC ATTCTATTCT GGGGGGTGGG | | |
| GTGGGGCAGG ACAGCAAGGG GGAGGATTGG | | |
| GAAGAGAATA GCAGGCATGC TGGGGAGGGC | | |
| CGCAGGAACC CCTAGTGATG GAGTTGGCCA | | |
| CTCCCTCTCT GCGCGCTCGC TCGCTCACTG | | |
| AGGCCGGGCG ACCAAAGGTC GCCCGACGCC | | |
| CGGGCTTTGC CCGGGCGGCC TCAGTGAGCG | | |
| AGCGAGCGCG CAGCTGCCTG CAGGGGCGCC | | |
| TGATGCGGTA TTTTCTCCTT ACGCATCTGT | | |
| GCGGTATTTC ACACCGCATA CGTCAAAGCA | | |
| ACCATAGTAC GCGCCCTGTA GCGGCGCATT | | |
| AAGCGCGGCG GGGGTGGTGG TTACGCGCAG | | |
| CGTGACCGCT ACACTTGCCA GCGCCTTAGC | | |
| GCCCGCTCCT TTCGCTTTCT TCCCTTCCTT | | |
| TCTCGCCACG TTCGCCGGCT TTCCCCGTCA | | |
| AGCTCTAAAT CGGGGGCTCC CTTTAGGGTT | | |
| CCGATTTAGT GCTTTACGGC ACCTCGACCC | | |
| CAAAAAACTT GATTTGGGTG ATGGTTCACG | | |
| TAGTGGGCCA TCGCCCTGAT AGACGGTTTT | | |
| TCGCCCTTTG ACGTTGGAGT CCACGTTCTT | | |
| TAATAGTGGA CTCTTGTTCC AAACTGGAAC | | |
| AACACTCAAC TCTATCTCGG GCTATTCTTT | | |
| TGATTTATAA GGGATTTTGC CGATTTCGGT | | |
| CTATTGGTTA AAAAATGAGC TGATTTAACA | | |
| AAAATTTAAC GCGAATTTTA ACAAAATATT | | |
| AACGTTTACA ATTTTATGGT GCACTCTCAG | | |
| TACAATCTGC TCTGATGCCG CATAGTTAAG | | |
| CCAGCCCCGA CACCCGCCAA CACCCGCTGA | | |
| CGCGCCCTGA CGGGCTTGTC TGCTCCCGGC | | |
| ATCCGCTTAC AGACAAGCTG TGACCGTCTC | | |
| CGGGAGCTGC ATGTGTCAGA GGTTTTCACC | | |
| GTCATCACCG AAACGCGCGA GACGAAAGGG | | |
| CCTCGTGATA CGCCTATTTT TATAGGTTAA | | |
| TGTCATGATA ATAATGGTTT CTTAGACGTC | | |
| AGGTGGCACT TTTCGGGGAA ATGTGCGCGG | | |
| AACCCCTATT TGTTTATTTT TCTAAATACA | | |
| TTCAAATATG TATCCGCTCA TGAGACAATA | | |
| ACCCTGATAA ATGCTTCAAT AATATTGAAA | | |
| AAGGAAGAGT | | |

### Methods

Disclosed herein are methods of treating a phosphoglucomutase 1 (PGM1) deficiency in a subject in need thereof. In some aspects, the methods can comprise administering to the subject a therapeutically effective amount of a recombinant adeno-associated virus (AAV) vector comprising an expression cassette comprising: a nucleic acid sequence encoding human phosphoglucomutase 1 (hPGM1), operably linked to one or more regulatory elements; and a polyadenylation tail signal; or pharmaceutical composition comprising any of the AAV vectors disclosed herein.

Disclosed herein are methods for the treatment and/or prevention of phosphoglucomutase 1 (PGM1) deficiency in a subject in a subject in need thereof. In some aspects, the methods can comprise administering to the subject any of the pharmaceutical compositions disclosed herein. In some aspects, the methods can comprise administering to the subject, any of the vector disclosed herein. In some aspects, the methods can comprise administering to the subject any of the polynucleotide disclosed herein.

Disclosed herein are methods for obtaining a recombinant adeno-associated viral vector (AAV) comprising a polynucleotide comprising an expression cassette, wherein the expression cassette comprises a transcriptional regulatory region comprising a promoter operatively linked to a nucleotide sequence as set forth in SEQ ID NO: 1 encoding the human phosphoglucomutase 1 (hPGM1) protein. In some aspects, the methods can comprise the steps of: (i) providing a cell comprising the polynucleotide comprising an expression cassette, wherein the expression cassette comprises a transcriptional regulatory region comprising a promoter operatively linked to a nucleotide sequence as set forth in SEQ ID NO: 1 encoding the human phosphoglucomutase 1 (hPGM1) protein, AAV cap proteins, AAV rep proteins and, optionally, viral proteins upon which AAV is dependent for replication, (ii) maintaining the cell under conditions adequate for assembly of the AAV; and (iii) purifying the adeno-associated viral vector produced by the cell.

Disclosed herein are methods of modulating glycosylation, glycogen metabolism or glycogen metabolism in a subject. Also disclosed herein are methods of normalizing glycosylation, glycogen metabolism or glycogen metabolism in a subject in need thereof. In some aspects, the methods can comprise administering to the subject a therapeutically effective amount of a recombinant adeno-associated virus (AAV) vector comprising an expression cassette comprising: a nucleic acid sequence encoding human phosphoglucomutase 1 (hPGM1), operably linked to one or more regulatory elements; and a polyadenylation tail signal; or pharmaceutical composition comprising any of the AAV vectors disclosed herein.

Disclosed herein are methods of increasing glucose metabolism in a subject. In some aspects, the methods can comprise administering to the subject a therapeutically effective amount of a recombinant adeno-associated virus (AAV) vector comprising an expression cassette comprising: a nucleic acid sequence encoding human phosphoglucomutase 1 (hPGM1), operably linked to one or more regulatory elements; and a polyadenylation tail signal; or pharmaceutical composition comprising any of the AAV vectors disclosed herein.

Disclosed herein are methods of increasing ejection fraction or fractional shortening in a subject. In some aspects, the methods can comprise administering to the subject a therapeutically effective amount of a recombinant adeno-associated virus (AAV) vector comprising an expression cassette comprising: a nucleic acid sequence encoding human phosphoglucomutase 1 (hPGM1), operably linked to one or more regulatory elements; and a polyadenylation tail signal; or pharmaceutical composition comprising any of the AAV vectors disclosed herein.

Disclosed herein are methods of reducing left ventricular mass in a subject. In some aspects, the methods can comprise administering to the subject a therapeutically effective amount of a recombinant adeno-associated virus (AAV) vector comprising an expression cassette comprising: a nucleic acid sequence encoding human phosphoglucomutase 1 (hPGM1), operably linked to one or more regulatory elements; and a polyadenylation tail signal; or pharmaceutical composition comprising any of the AAV vectors disclosed herein.

Disclosed herein are methods of reducing the early (E) to late (A) ventricular filing velocities (E/A ratio) in a subject. In some aspects, the methods can comprise administering to the subject a therapeutically effective amount of a recombinant adeno-associated virus (AAV) vector comprising an expression cassette comprising: a nucleic acid sequence encoding human phosphoglucomutase 1 (hPGM1), operably linked to one or more regulatory elements; and a polyadenylation tail signal; or pharmaceutical composition comprising any of the AAV vectors disclosed herein.

Disclosed herein are methods of reducing a disease condition in a subject suffering from PGM1-CDG. In some aspects, the disease condition can be hyptonia, hypoglycemia, cardiomyopathy, growth retardation, hormonal deficiencies, myopathy, hepatopathy, hypogonadotropic hypogonadism, malignant hyperthermia, o dioder or a combination thereof. In some aspects, the methods can comprise administering to the subject a therapeutically effective amount of a recombinant adeno-associated virus (AAV) vector comprising an expression cassette comprising: a nucleic acid sequence encoding human phosphoglucomutase 1 (hPGM1), operably linked to one or more regulatory elements; and a polyadenylation tail signal; or pharmaceutical composition comprising any of the AAV vectors disclosed herein.

Disclosed herein are methods of treating a subject suffering from PGM1 deficiency by administration of an rAAV comprising a transgene encoding PGM1 and engineered to express the PGM1 protein in the heart and other tissue(s), in particular an rAAV9 vector comprising, for example, the construct disclosed herein, such as comprising the nucleotide sequence of SEQ ID NO: 1. The rAAV encoding the PGM1 protein can be administered by any method known in the art. In some aspects, the rAAV can delivered by intravenous, intra-arterial, intramuscular, intracardiac, intraperitoneal, subcutaneous, or inhalation administration. In some aspects, methods for delivering a transgene to heart tissue in a subject can comprise co-administering of an effective amount of a rAAV by two different administration routes, e.g., by intracardiac administration and by intravenous administration. Co-administration of the rAAV can be performed at approximately the same time, or different times.

The dose of rAAV to be administered in methods disclosed herein will vary depending, for example, on the particular rAAV, the mode of administration, the treatment goal, the individual, and the cell type(s) being targeted, and may be determined by methods standard in the art. Titers of each rAAV administered may range from about 1×10⁶, about 1×10⁷, about 1×10⁸, about 1×10⁹, about 1×10¹⁰, about 1×10¹¹, about 1×10¹², about 1×10¹³, about 1×10¹⁴, or to about 1×10¹⁵ or more DNase resistant particles (DRP) per ml. Dosages may also be expressed in units of viral genomes (vg) (i.e., 1×10⁷ vg, 1×10⁸ vg, 1×10⁹ vg, 1×10¹⁰ vg, 1×10¹¹ vg, 1×10¹² vg, 1×10¹³ vg, 1×10¹⁴ vg, 1×10¹⁵, respectively). Dosages may also be expressed in units of viral genomes (vg) per kilogram (kg) of bodyweight (i.e., 1×10¹⁰ vg/kg, 1×10¹¹ vg/kg, 1×10¹² vg/kg, 1×10¹³ vg/kg, 1×10¹⁴ vg/kg, 1×10¹⁵ vg/kg respectively). Methods for titering AAV are described in Clark et al., Hum. Gene Ther., 10: 1031-1039 (1999).

The combination of the rAAV serotype, including AAV9, the regulatory elements, and mode of administration result in therapeutically effective delivery of the PGM1 protein to heart tissues as well as other peripheral tissues that promote the therapeutic benefit of the administration.

In any of the methods disclosed herein, the subject has a PGM1 deficiency or has been diagnosed with PGM1-CDG.

In some aspects, the heart tissue to be targeted can be cardiomyocytes. In some aspects, the brain, liver, muscle, heart or a combination thereof can be targeted. The administration route for targeting heart tissue can depend on the AAV serotype. In some aspects, the administration route can be intravascular injection when the AAV serotype is AAVPHP.B, AAV1, AAV6, AAV6.2, AAV7, AAV8, AAV9, AAVrh.10, rh.39, rh.43 and CSp3. In some aspects, the administration route can be ICM administration when the AAV serotype is AAVPHP.B, AAV1, AAV6, AAV6.2, AAV7, AAV8, AAV9, AAVrh.10, rh.39, rh.43 and CSp3.

In some aspects, the composition (e.g., a pharmaceutical composition) can comprise an rAAV comprising a nucleic acid encoding a PGM1. In some aspects, the compositions comprising a recombinant AAV comprising at least one modified genetic regulatory sequence or element can further comprise a pharmaceutically acceptable carrier. Suitable carriers can be selected for the indication for which the rAAV is directed. For example, one suitable carrier includes saline, which may be formulated with a variety of buffering solutions (e.g., phosphate buffered saline). Examples of other suitable carriers include but are not limited to sterile saline, lactose, sucrose, calcium phosphate, gelatin, dextran, agar, pectin, peanut oil, sesame oil, and water. Optionally, the compositions disclosed herein can also include, in addition to the rAAV and carrier(s), other pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

In some aspects, the rAAV can be administered in a pharmaceutical composition comprising phosphate buffered saline (PBS), pH 7.3 and 0.001% of a pharmaceutically acceptable non-ionic surfactant, such as, for example, pluronic F-68 (PF68), or other appropriate pharmaceutically acceptable buffers or excipients. The formulation may be frozen until ready for use and then thawed and administered.

In some aspects, the compositions disclosed herein can comprise an rAAV alone, or in combination with one or more other viruses (e.g., a second rAAV encoding having one or more different transgenes). In some aspects, a composition can comprise 1, 2, 3, 4, 5, 6, 7, 8, 9, 10, or more different rAAVs each having one or more different transgenes.

rAAVs can be administered in sufficient amounts to transfect the cells of a desired tissue and to provide sufficient levels of gene transfer and expression without undue adverse effects. In some aspects, acceptable routes of administration include, but are not limited to, direct delivery to the selected organ (e.g., injection into the liver, skeletal muscle, heart), oral, intravenous, intramuscular, subcutaneous, intradermal, intratumoral, and other parental routes of administration. In some aspects, the route of administration can be by direct injection. In some aspects, the route of administration can be by intravenous delivery. Routes of administration can be combined, if desired.

The dose of rAAV virions required to achieve a particular “therapeutic effect,” e.g., the units of dose in genome copies/per kilogram of body weight (GC/kg), the units of dose in genome copies per heart volume, will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine a rAAV virion dose range to treat a patient having a particular disease or disorder based on the aforementioned factors, as well as other factors that are well known in the art.

An effective amount of an rAAV is an amount sufficient to target infect an animal, target a desired tissue. The effective amount will depend primarily on factors such as the species, age, weight, health of the subject, and the tissue to be targeted, and may thus vary among animal and tissue. For example, an effective amount of the rAAV can be in the range from about 1 ml to about 100 ml of solution containing from about 10⁶ to 10¹⁶ genome copies (e.g., from 1×10⁶ to 1×10¹⁶, inclusive). In methods disclosed herein, the therapeutically effective dose is between 6×10¹³ gc/kg to 6×10¹⁴ gc/kg, including 7×10¹³ gc/kg, 8×10¹³ gc/kg, 9×10¹³ gc/kg, 1×10¹⁴ gc/kg, 2×10¹⁴ gc/kg, 3×10¹⁴ gc/kg, 4×10¹⁴ gc/kg, or 5×10¹⁴ gc/kg (or alternatively, genome copies per heart volume or other measurement appropriate for intracardiac delivery). In some aspects, a dosage between about 10¹¹ to 10¹² per kg or appropriate measurement rAAV genome copies can be appropriate. In some aspects, a dosage of between about 10¹¹ to 10¹³ per kg or appropriate measurement rAAV genome copies can be appropriate. In some aspects, a dosage of between about 10¹¹ to 10¹⁴ per kg or appropriate measurement rAAV genome copies can be appropriate. In some aspects, a dosage of between about 10¹¹ to 10¹⁵ per kg or appropriate measurement rAAV genome copies can be appropriate. In some aspects, a dosage of about 1×10¹⁴ vector genome (vg) copies per kg or appropriate measurement can be appropriate. In some aspects, the dosage can vary or be reduced when specifically targeting one or more organs (e.g., liver, brain, skeletal muscle, or heart). In some aspects, a dosage between about 107 to 108 rAAV genome copies per kg or appropriate measurement can be appropriate. In some aspects, a dosage of between about 108 to 10′ rAAV genome copies per kg or appropriate measurement can be appropriate. In some aspects, a dosage of between about 10′ to 10¹⁰ rAAV genome copies per kg or appropriate measurement can be appropriate. In some aspects, a dosage of between about 10¹⁰ to 10¹¹ rAAV genome copies per kg or other appropriate measurement can be appropriate.

In some aspects, a potential side-effect for administering an AAV to a subject can be an immune response in the subject to the AAV, including inflammation, and, and may depend on the route of administration, and in particularly, when the administration of an AAV is systemic. In some aspects, a subject can be immunosuppressed prior to administration of one or more rAAVs as described herein.

As used herein, “immunosuppressed” or “immunosuppression” refers to a decrease in the activation or efficacy of an immune response in a subject. Immunosuppression can be induced in a subject using one or more (e.g., multiple, such as 2, 3, 4, 5, or more) agents, including, but not limited to, rituximab, methylprednisolone, prednisolone, sirolimus, immunoglobulin injection, prednisone, methotrexate, and any combination thereof.

In some aspects, methods disclosed herein can further comprise the step of inducing immunosuppression (e.g., administering one or more immunosuppressive agents) in a subject prior to the subject being administered an rAAV (e.g., an rAAV or pharmaceutical composition as disclosed herein). In some aspects, a subject can be immunosuppressed (e.g., immunosuppression is induced in the subject) between about 30 days and about 0 days (e.g., any time between 30 days until administration of the rAAV, inclusive) prior to administration of the rAAV to the subject. In some aspects, the subject can be pretreated with immune suppression agent (e.g., rituximab, sirolimus, and/or prednisone) for at least 7 days. In some aspects, immunosuppression of a subject maintained during and/or after administration of a rAAV or pharmaceutical composition. In some aspects, a subject can be immunosuppressed (e.g., administered one or more immunosuppressants) for between 1 day and 1 year after administration of the rAAV or pharmaceutical composition.

In some aspects, rAAV compositions can be formulated to reduce aggregation of AAV particles in the composition, particularly where high rAAV concentrations are present (e.g., −10¹³ GC/ml or more). Methods for reducing aggregation of rAAVs are well known in the art and, include, for example, addition of surfactants, pH adjustment, salt concentration adjustment, etc. (See, e.g., Wright F R, et al., Molecular Therapy (2005) 12, 171-178, the contents of which are incorporated herein by reference.)

Formulation of pharmaceutically-acceptable excipients and carrier solutions are well-known to those of skill in the art, as is the development of suitable dosing and treatment regimens for using the particular compositions described herein in a variety of treatment regimens.

In some aspects, these formulations can contain at least about 0.1% of the active compound or more, although the percentage of the active ingredient(s) may, of course, be varied and can be conveniently be between about 1 or 2% and about 70% or 80% or more of the weight or volume of the total formulation. Naturally, the amount of active compound in each therapeutically-useful composition can be prepared in such a way that a suitable dosage can be obtained in any given unit dose of the compound. Factors such as solubility, bioavailability, biological half-life, route of administration, product shelf life, as well as other pharmacological considerations can be contemplated by one skilled in the art of preparing such pharmaceutical formulations, and as such, a variety of dosages and treatment regimens can be desirable.

In some aspects, it will be desirable to deliver the rAAV-based therapeutic constructs in suitably formulated pharmaceutical compositions as disclosed herein either subcutaneously, intrapancreatically, intranasally, intracardiacally, parenterally, intravenously, intramuscularly, or orally, intraperitoneally, or by inhalation. In some aspects, the administration modalities as described in U.S. Pat. Nos. 5,543,158; 5,641,515 and 5,399,363 (each specifically incorporated herein by reference in its entirety) can be used to deliver rAAVs. In some aspects, a preferred mode of administration can be intravenous delivery.

The pharmaceutical forms suitable for injectable use include sterile aqueous solutions or dispersions and sterile powders for the extemporaneous preparation of sterile injectable solutions or dispersions. Dispersions can also be prepared in glycerol, liquid polyethylene glycols, and mixtures thereof and in oils. Under ordinary conditions of storage and use, these preparations contain a preservative to prevent the growth of microorganisms. In many cases the form can be sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, isotonic agents, for example, sugars or sodium chloride can be included. Prolonged absorption of the injectable compositions can be brought about by the use in the compositions of agents delaying absorption, for example, aluminum monostearate and gelatin.

For administration of an injectable aqueous solution, for example, the solution can be suitably buffered, if necessary, and the liquid diluent first rendered isotonic with sufficient saline or glucose. These particular aqueous solutions can be suitable for intravenous, intramuscular, subcutaneous, and intraperitoneal administration. In this connection, a sterile aqueous medium that can be employed will be known to those of skill in the art. For example, one dosage can be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). In particular embodiments, the rAAV is formulated in phosphate buffered saline (PBS) at pH 7.3, including 0.001% of a pharmaceutically acceptable non-ionic surfactant, such as, for example, PF68. Some variation in dosage will necessarily occur depending on the condition of the host. The person responsible for administration will, in any event, determine the appropriate dose for the individual host.

Sterile injectable solutions can be prepared by incorporating the active rAAV in the required amount in the appropriate solvent with various of the other ingredients enumerated herein, as required, followed by filtered sterilization. Generally, dispersions can be prepared by incorporating the various sterilized active ingredients into a sterile vehicle which contains the basic dispersion medium and the required other ingredients from those enumerated above. In the case of sterile powders for the preparation of sterile injectable solutions, the methods of preparation can be vacuum-drying and freeze-drying techniques which yield a powder of the active ingredient plus any additional desired ingredient from a previously sterile-filtered solution thereof.

The rAAV compositions disclosed herein can be also be formulated in a neutral or salt form. Pharmaceutically-acceptable salts, include the acid addition salts (formed with the free amino groups of the protein) and which can be formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations can be easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the rAAV vector delivered transgenes can be formulated for delivery either encapsulated in a lipid particle, a liposome, a vesicle, a nanosphere, or a nanoparticle or the like.

Such formulations can be used for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes can be formed from phospholipids that can be dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Angstroms, containing an aqueous solution in the core. Alternatively, nanocapsule formulations of the rAAV can be used. Nanocapsules can generally entrap substances in a stable and reproducible way. To avoid side effects due to intracellular polymeric overloading, such ultrafine particles (sized around 0.1 p.m.) should be designed using polymers able to be degraded in vivo. Biodegradable polyalkyl-cyanoacrylate nanoparticles that meet these requirements are contemplated for use.

In addition to the methods of delivery described above, the following techniques can also be used as alternative methods of delivering the rAAV compositions to a host. Sonophoresis (e.g., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

In some aspects, the methods can include administering one or more additional therapeutic agents to a subject who has been administered an rAAV or pharmaceutical composition as described herein.

Disclosed herein are methods of treating a PGMI deficiency by administration of an rAAV vector described herein that contains a transgene encoding PGM1 engineered to be expressed in the heart, and can be an AAV9 serotype. PGM1 deficiency, which results from loss-of-function mutations in the PGM1 gene is an ultra-rare genetic disorder, and patients suffer from hyptonia, hypoglycemia, cardiomyopathy, growth retardation, hormonal deficiencies, myopathy, hypogonadotropic hypogonadism, rialignant hyperthermia, disorder or hepatopathy. In some aspects, gene replacement therapy as described herein that can be useful to restore PGM1 function, in one or more tissues (or organs) including liver, brain, skeletal muscle and heart, which can alleviate the disease symptoms.

Disclosed herein are isolated nucleic acids, rAAVs, compositions, and methods useful in treating PGM1 Deficiency. In some aspects, the methods for treating PGM1 deficiency in a subject can comprise administering an rAAV that contains a transgene encoding PGM1, for example having a coding sequence of SEQ ID NO: 1, in a gene expression cassette engineered to express the PGM1 in the heart (for example under the control of a CAG promoter, for example, the construct having the nucleotide sequence of SEQ ID NO: 8 (including the nucleotide sequence of SEQ ID NO: 1 operably linked to a CAG promoter and a polyA signal sequence) or SEQ ID NO: 9 (the entire construct with the flanking ITR sequences)) and the rAAV is an AAV9 serotype. In some aspects, the rAAV can be administered by direct injection (e.g., intracardiacally). In some aspects, the rAAV can be administered intramuscularly, intravenously or intracardiacally.

Also disclosed herein are methods of promoting expression of functional PGM1 protein in a subject (e.g., in the heart and in other tissues of a subject) comprising administering, including intramuscular, intravenous or intracardiac administration, the rAAVs described herein to a subject having or suspected of having a disease of disorder associated with low levels of PGM1 expression (e.g., PGM1 deficiency). As used herein, a disease of disorder associated with low levels of PGM1 expression is a disease or disorder in which a subject has at least 10%, at least 20%, at least 30%, at least 40%, at least 50%, at least 60%, at least 70%, at least 80%, or at least 90% lower levels of PGM1 expression relative to a control subject (e.g., a healthy subject or an untreated subject).

In some aspects, administering the rAAVs described herein to a subject promotes expression of PGM1 by between 2-fold and 100-fold (e.g., 2-fold, 5-fold, 10-fold, 20-fold, 50-fold, 75-fold, 100-fold, etc.) compared to a control subject. In some aspects, administering the rAAVs described herein to a subject promotes expression of PGM1 in the heart of a subject by between 2-fold and 100-fold (e.g., 2-fold, 5-fold, 10-fold, 20-fold, 50-fold, 75-fold, 100-fold, etc.) compared to a control subject. As used herein a “control” subject may refer to a subject that is not administered the isolated nucleic acids, the rAAVs, or the compositions described herein or a healthy subject. In some aspects, a control subject can be the same subject that is administered the isolated nucleic acids, the rAAVs, or the compositions described herein (e.g., prior to the administration). In some aspects, administering the isolated nucleic acids, the rAAVs, or the compositions described to a subject promotes expression of PGM1 by 2-fold compared to a control. In some aspects, administering the rAAVs described to a subject promotes expression of PGM1 by 100-fold compared to a control. In some aspects, administering the rAAVs described to a subject promotes expression of PGM1 by 5-fold compared to a control. In some aspects, administering the rAAVs described to a subject promotes expression of PGM1 by 10-fold compared to a control. In some aspects, administering the rAAVs described herein to a subject promotes expression of PGM1 by 5-fold to 100-fold compared to control (e.g., 5-fold to 10-fold, 10-fold to 15-fold, 10-fold to 20-fold, 15-fold to 25-fold, 20-fold to 30-fold, 25-fold to 35-fold, 30-fold to 40-fold, 35-fold to 45-fold, 40-fold to 60-fold, 50-fold to 75-fold, 60-fold to 80-fold, 75-fold to 100-fold compared to a control).

In some aspects, administering the rAAVs described herein to a subject promotes expression of PGM1 in a subject (e.g., promotes expression of PGM1 in the heart, liver, brain, skeletal muscle or a combination thereof of a subject) by between a 5% and 200% increase (e.g., 5-50%, 25-75%, 50-100%, 75-125%, 100-200%, or 100-150% etc.) compared to a control subject.

Further disclosed herein are methods of treating a subject having a disease of disorder associated with low levels of PGM1 expression (e.g., PGM1 deficiency). In some aspects, the methods can comprise administering to the subject an effective amount of an rAAV comprising a capsid containing a nucleic acid engineered to express PGM1 in the heart, liver, brain, skeletal muscle or a combination thereof of the subject particularly by intramuscular, intravenous or intracardiac administration. As used herein, the term “treating” refers to the application or administration of a composition (e.g., an isolated nucleic acid or rAAV as described herein) to a subject who has a disease or disorder associated with low levels of PGM1 expression (e.g., PGM1 deficiency), with the purpose to cure, heal, alleviate, relieve, alter, remedy, ameliorate, improve, or affect the disorder, the symptom of the disease, or the predisposition toward a disease. Alleviating a disease associated with low levels of PGM1 expression (e.g., PG,1 deficiency) includes delaying the development or progression of the disease, or reducing disease severity. Alleviating the disease does not necessarily require curative results. As used therein, “delaying” the development of a disease means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

In particular, administration of the rAAV described herein to a human subject suffering from PGM1 deficiency will within 10 weeks, 15 weeks, 20 weeks, 25 weeks, 30 weeks, 40 weeks, 50 weeks or 1 year after the administration will result in reduction in one or more biomarkers or hallmarks of the disease. In particular, administration of the rAAV described herein to a human subject suffering from PGM1 deficiency will result in improvements in heart functions (as measured by an echocardiography), developmental delay, reduction in levels of liver transaminases, and improvements in biomarkers including but not limited to transferrin glycosylation and coagulation times.

“Development” or “progression” of a disease means initial manifestations and/or ensuing progression of the disease. Development of the disease can be detectable and assessed using standard clinical techniques as well known in the art. However, development also refers to progression that can be undetectable. As used herein the terms development or progression refer to the biological course of the symptoms. “Development” includes occurrence, recurrence, and onset. As used herein “onset” or “occurrence” of a disease can be associated with low levels of PGM1 expression (e.g., PGM1 deficiency).

In some aspects, the subject can be a human, a mouse, a rat, a pig, a dog, a cat, or a non-human primate. In some aspects, a subject has or is suspected of having a disease or disorder associated with low levels of PGM1 expression (e.g., PGM1 deficiency).

In some aspects, the rAAVs disclosed herein can be administered in sufficient amounts to transfect the cells of a desired tissue and to provide sufficient levels of gene transfer and expression without undue adverse effects. Pharmaceutically acceptable routes of administration include, but are not limited to, direct delivery to the selected organ (e.g., to the heart), oral, inhalation (including intranasal and intratracheal delivery), intravenous, intramuscular, subcutaneous, intradermal, and other parental routes of administration. Routes of administration can be combined, if desired.

In some aspects, the dose of rAAV virions required to achieve a particular “therapeutic effect,” e.g., the units of dose in genome copies/per kilogram of body weight (GC/kg) (or alternatively based upon heart size or heart volume), can vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine a rAAV virion dose range to treat a patient having a particular disease or disorder based on the aforementioned factors, as well as other factors that are well known in the art. An effective amount of an rAAV is an amount sufficient to target infect a subject or target a desired tissue. In some aspects, an effective amount of an rAAV is an amount sufficient to produce a stable somatic transgenic animal model. The effective amount will depend primarily on factors such as the species, age, weight, health of the subject, and the tissue to be targeted, and may thus vary among animal and tissue. For example, an effective amount of the rAAV can be in the range of from about 1 ml to about 100 ml of solution containing from about 10¹ to 10¹⁶ genome copies. In some aspects, the rAAV transduces hepatocytes or cardiomyocytes. In some aspects, the rAAV transduces cells in the brain. In some aspects, the rAAV transduces skeletal muscle cells. In some aspects, the rAAV transduces cells in the heart, brain, liver, skeletal muscle or a combination thereof. In some aspects, the effective amount of rAAV can be 10¹⁰, 10¹¹, 10¹², 10¹³, or 10¹⁴ genome copies per kg. In some aspects, the effective amount of rAAV can be 10¹⁰, 10¹¹, 10¹², 10¹³, 10¹⁴, or 10¹⁵ genome copies per subject. In some cases, a dosage between about 6×10⁰⁹ to 6×10¹⁴ rAAV genome copies can be appropriate.

In some aspects, rAAV compositions can be formulated to reduce aggregation of AAV particles in the composition, particularly where high rAAV concentrations are present (e.g., −10¹³ GC/ml or more). Methods for reducing aggregation of rAAVs are well known in the art and, include, for example, addition of surfactants, pH adjustment, salt concentration adjustment, etc. (See, e.g., Wright F R, et al., Molecular Therapy (2005) 12, 171-178, the contents of which are incorporated herein by reference.)

Assessment of Therapeutic Efficacy. The efficacy of the rAAV compositions described herein may be assessed by in vitro assays and by in vivo assays, for example in PGM1 deficiency animal models. Assessment of efficacy of administration is described in Example 1.

### Kits

Disclosed herein are kits comprising any of the agents described herein. In some aspects, any of the agents disclosed herein can be assembled into pharmaceutical or diagnostic or research kits to facilitate their use in therapeutic, diagnostic or research applications. A kit can include one or more containers housing the components of the disclosure and instructions for use. Specifically, such kits may include one or more agents described herein, along with instructions describing the intended application and the proper use of these agents. In some aspects, the agents in a kit can be in a pharmaceutical formulation and dosage suitable for a particular application and for a method of administration of the agents. Kits for research purposes can contain the components in appropriate concentrations or quantities for running various experiments.

Also disclosed herein are kits for producing a rAAV. In some aspects, the kit can comprise a container housing an isolated nucleic acid encoding a PGM1 protein or a portion thereof. In some aspects, the kits can further comprise instructions for producing the rAAV. In some aspects, the kit further comprises at least one container housing a recombinant AAV vector, wherein the recombinant AAV vector comprises a transgene.

In some aspects, the kits can comprise a container housing a recombinant AAV as described supra. In some aspects, the kits can further comprises a container housing a pharmaceutically acceptable carrier. For example, a kit can comprise one container housing a rAAV and a second container housing a buffer suitable for injection of the rAAV into a subject. In some aspects, the container can be a syringe.

In some aspects, the kits can be designed to facilitate use of the methods described herein by researchers and can take many forms. Each of the compositions of the kit, where applicable, may be provided in liquid form (e.g., in solution), or in solid form, (e.g., a dry powder). In some aspects, some of the compositions can be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions can be associated with the kit, for example, audiovisual (e.g., videotape, DVD, etc.), Internet, and/or web-based communications, etc. The written instructions can be in a form prescribed by a governmental agency regulating the manufacture, use or sale of pharmaceuticals or biological products, which instructions can also reflect approval by the agency of manufacture, use or sale for animal administration.

The kits disclosed herein can also contain any one or more of the components described herein in one or more containers. In some aspects, the kits can include instructions for mixing one or more components of the kit and/or isolating and mixing a sample and applying to a subject. The kits can include a container housing agents described herein. The agents can be in the form of a liquid, gel or solid (powder). The agents can be prepared sterilely, packaged in syringe and shipped refrigerated. Alternatively, it can be housed in a vial or other container for storage. A second container can have other agents prepared sterilely. Alternatively the kits can include the active agents premixed and shipped in a syringe, vial, tube, or other container. The kits can have one or more or all of the components required to administer the agents to an animal, such as a syringe, topical application devices, or iv needle tubing and bag, particularly in the case of the kits for producing specific somatic animal models.

In some aspects, the method disclosed herein can involve transfecting cells with total cellular DNAs isolated from the tissues that potentially harbor proviral AAV genomes at very low abundance and supplementing with helper virus function (e.g., adenovirus) to trigger and/or boost AAV rep and cap gene transcription in the transfected cell. In some aspects, RNA from the transfected cells can provide a template for RT-PCR amplification of cDNA and the detection of novel AAVs. In cases where cells are transfected with total cellular DNAs isolated from the tissues that potentially harbor proviral AAV genomes, it is often desirable to supplement the cells with factors that promote AAV gene transcription. For example, the cells can also be infected with a helper virus, such as an Adenovirus or a Herpes Virus. In some aspects, the helper functions can be provided by an adenovirus. The adenovirus can be a wild-type adenovirus, and can be of human or non-human origin, for example, non-human primate (NHP) origin. Similarly, adenoviruses known to infect non-human animals (e.g., chimpanzees, mouse) can also be employed in the methods of the disclosure (See, e.g., U.S. Pat. No. 6,083,716). In addition to wild-type adenoviruses, recombinant viruses or non-viral vectors (e.g., plasmids, episomes, etc.) carrying the necessary helper functions can be utilized. Such recombinant viruses are known in the art and may be prepared according to published techniques. See, e.g., U.S. Pat. Nos. 5,871,982 and 6,251,677, which describe a hybrid Ad/AAV virus. A variety of adenovirus strains are available from the American Type Culture Collection, Manassas, Va., or available by request from a variety of commercial and institutional sources. Further, the sequences of many such strains are available from a variety of databases including, e.g., PubMed and GenBank.

Cells can also be transfected with a vector (e.g., helper vector) which provides helper functions to the AAV. The vector providing helper functions can provide adenovirus functions, including, e.g., Ela, Elb, E2a, E40RF6. The sequences of adenovirus gene providing these functions can be obtained from any known adenovirus serotype, such as serotypes 2, 3, 4, 7, 12 and 40, and further including any of the presently identified human types known in the art. Thus, in some aspects, the methods involve transfecting the cell with a vector expressing one or more genes necessary for AAV replication, AAV gene transcription, and/or AAV packaging.

In some aspects, an isolated capsid gene can be used to construct and package recombinant AAV vectors, using methods well known in the art, to determine functional characteristics associated with the novel capsid protein encoded by the gene. For example, isolated capsid genes can be used to construct and package recombinant AAV (rAAV) vectors comprising a reporter gene (e.g., B-Galactosidase, GFP, Luciferase, etc.). The rAAV vector can then be delivered to an animal (e.g., mouse) and the tissue targeting properties of the isolated capsid gene can be determined by examining the expression of the reporter gene in various tissues (e.g., heart, liver, kidneys) of the animal. Other methods for characterizing isolated capsid genes are disclosed herein and still others are well known in the art.

The kits disclosed can have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kits can be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kits can also include other components, depending on the specific application, for example, containers, cell media, salts, buffers, reagents, syringes, needles, a fabric, such as gauze, for applying or removing a disinfecting agent, disposable gloves, a support for the agents prior to administration etc. The instructions included within the kit can involve methods for detecting a latent AAV in a cell. In addition, kits of the disclosure can include, instructions, a negative and/or positive control, containers, diluents and buffers for the sample, sample preparation tubes and a printed or electronic table of reference AAV sequence for sequence comparisons.

### EXAMPLES

### Example 1: AAV9-hPGM1 Gene Replacement Therapy Prevents the Manifestation DCM in Pgm2 cKO Mice

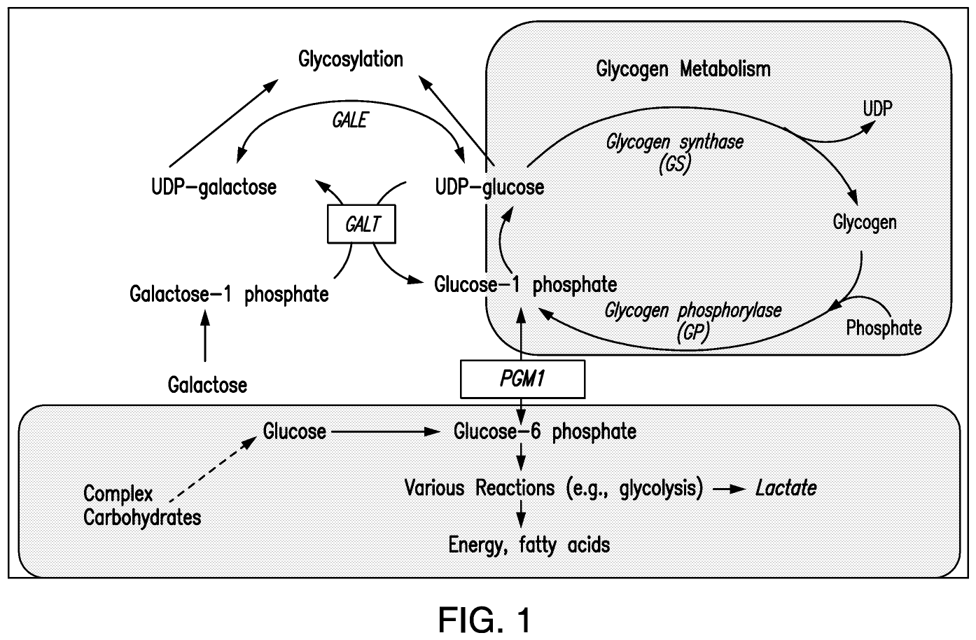

Phosphoglucomutase 1 (PGM1, FIG. 1) catalyzes the interconversion of glucose-1 phosphate (Glc-1P) and glucose-6 phosphate (Glc-6P) and therefore, it plays a fundamental role in glycolysis, glycogenesis, and glycogenolysis. PGM1 deficiency resulting from biallelic pathogenic variants in the human PGM1 genes leads to Glycogen Storage Disorder (GSD) Type 14. However, it has lately been revealed that PGM1 deficiency is also a Congenital Disorders of Glycosylation (CDG) characterized by both Type I & II glycosylation defects, resulting in both missing and truncated, hypogalactosylated glycans. In addition to the characteristic liver, skeletomuscular, endocrine and coagulation involvement, the most life-threatening complication of PGM1-CDG is the early-onset dilated cardiomyopathy (DCM). The precise pathophysiology of these tissue-specific disease phenotypes remains unclear. Recently, it has been shown that oral D-galactose supplementation in patients with PGM1 deficiency improves serum transferrin hypo-glycosylation, liver function, endocrine abnormalities, and reduced frequency of hypoglycemic episodes. Yet, it is also clear that not all clinical abnormalities, which include the deadly dilated cardiomyopathy and myopathy, were corrected. Therefore, the pathophysiology of the disease is may be tissue-specific. In this study, a cardiomyocyte-specific conditional Pgm2 (mouse ortholog of human PGM1) gene knockout (KO) mouse model was constructed and characterized at multiple levels. The results demonstrated that the animal model recapitulates many of the patient phenotypes, and also provides information about the pathobiology of the cardiac phenotype of the human disease. Finally, the efficacy of PGM1 augmentation in the animal model via AAV9-PGM1 gene replacement therapy was investigated and the results showed that it can prevent and halt the progression of the disease phenotype.

Methods. Animal models. Pgm2^{fl/fl} mice were constructed and developed at the University of Utah Mutagenesis Generation and Detection Core Facility and the University of Utah Transgenic and Gene Knockout Core Facility.

αMHC-MerCreMer transgenic mice were also used.

Genotyping. Genomic DNA was prepared from tail clips using the Qiagen Blood and tissue DNA isolation kit (Qiagen) according to the manufacturer's protocol. Approximately 10 ng of the genomic DNA was used for PCR. Genotyping of Myh6-MCM mice was performed using 2 sets of primers. The first primer set was designed to amplify the Cre Tg construct. The second primer set was used to amplify a mouse WT internal positive control. Genotyping of Pgm2^{fl/fl} mice was performed by PCR amplification.

AAV Vectors. AAV-hPGM1 contained a CAG promoter driving codon optimized human full-length PGM1 cDNA was synthesized by VectorBuilder Inc (Chicago, U.S.A.) on a fee-for-service basis.

Echocardiography. Cardiac functions of the mice were non-invasively monitored by transthoracic echocardiography using a Visual Sonics Vevo 2100 high-resolution imaging system with a 30 MHz scan head, 1, 2, 3, 4 and 8 weeks after oral administration of tamoxifen. Parasternal short- and long-axis views were used to obtain 2D and M-mode images. At least 10 independent cardiac cycles per experiment were obtained.

Histological analysis. Hearts were fixed in 10% formalin (PBS buffered), dehydrated, and embedded in paraffin. Heart architecture was determined using transverse 5-μm deparaffinized sections stained with H&E. Fibrosis was detected with Masson trichrome staining. Oil red O staining was performed on sections of unfixed, freshly frozen heart samples (6-μm in thickness).

Electron microscopy. Cardiac tissues from the left ventricle free wall was fixed with 2% glutaraldehyde in sodium cacodylate buffer at 4° C. Fixed tissues were embedded in epoxy resin, processed for thin (70-nm) cutting with a Leica UC6 ultramicrotome, and examined with a FEI Tecnai 12 Transmission Electron Microscope.

Western blot analysis. For Western blotting of total proteins, hearts were homogenized in a modified RIPA buffer containing protease and phosphatase inhibitors (Roche). Total protein lysates were isolated from hearts in RIPA buffer containing protease and phosphatase inhibitors. Samples were run on an SDS-PAGE gel and transferred to a nitrocellulose membrane (Invitrogen). Antibodies against PGM1 protein was purchased from Sigma, and anti-GAPDH antibodies were purchased from Cell Signaling Technology Inc. The protein bands were developed with an enhanced chemiluminescence substrate kit/IR dye. Quantification of blots was performed using ImageJ software (NIH).

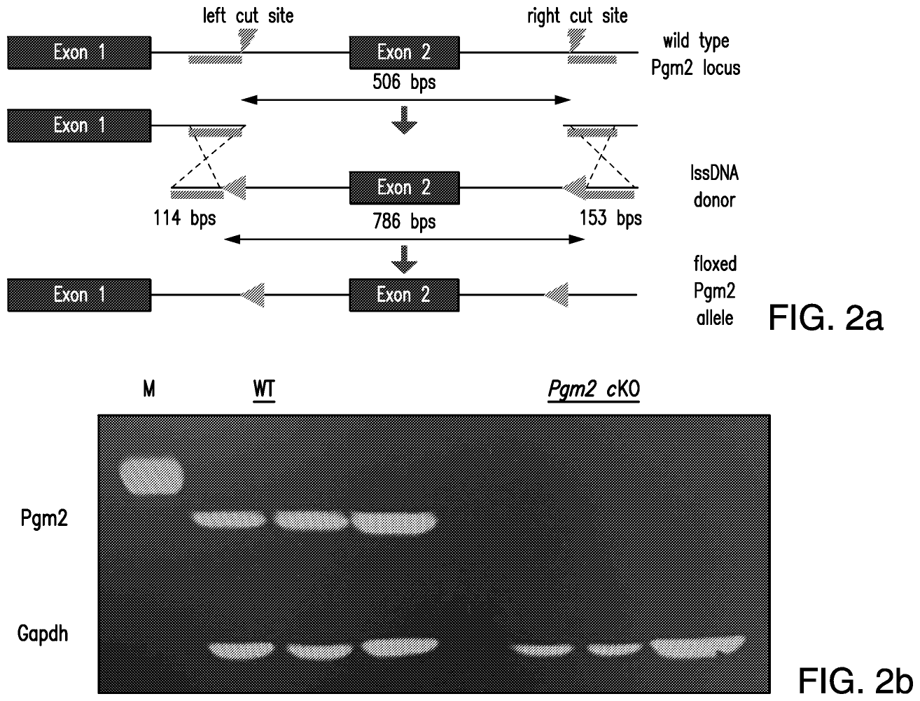

Results. Construction of a new tamoxifen-inducible cardiomyocyte-specific Pgm2 knockout (Pgm2 cKO) mouse model. Due to limited research utility of the constitutive Pgm2 KO mouse model, which is embryonic lethal, a tissue-specific conditional Pgm2 gene-KO mouse model was constructed in order to gain investigate the role of Pgm2 in tissue-specific functions in a living mammal. In particular, a tamoxifen-inducible cardiomyocyte-specific Pgm2 cKO mouse model was used. To develop this animal model, Pgm2-floxed animals were constructed. Briefly, validated CRISPR-Cas9 RNP complexes along with a long ssDNA donor that inserted two LoxP sites in the introns flanking exon 2 of Pgm2 (FIG. 2a), were microinjected into the male pronuclei of fertilized C57BL6 embryos. Injected embryos were implanted into pseudo pregnant mothers and carried to term. Offspring were genotyped using PCR-Restriction Enzyme Digest assays that identified the insertion of the donor DNA. Offspring showing insertion were then sequenced to confirm the perfect insertion of the donor DNA, including functional LoxP sites (FIG. 2A). Homozygous Pgm2-floxed animals were subsequently obtained by crossing heterozygous parents. The homozygous Pgm2-floxed animals were crossed to mice that harbored the alpha-MHC-MerCreMer (αMHC-MerCreMer) transgene, which has the mouse cardiac-specific alpha-myosin heavy chain promoter (αMHC or alpha-MHC; Myh6) directing the expression of a tamoxifen-inducible Cre recombinase (MerCreMer) in juvenile and adult cardiac myocytes. These αMHC-MerCreMer transgenic mice allow the creation of bi-transgenic mice for Cre-lox studies of temporally regulated deletion of loxP-flanked targeted genes in cardiac tissues/cells. Once the progeny with the desired combined genotypes were obtained and confirmed, oral treatment of the animals started with tamoxifen (35 mg/kg body weight for 5 days) at approximately 4 weeks of age in order to induce the excision of exon 2 of the Pgm2 alleles in the cardiomyocytes. Oral administration of tamoxifen was chosen because it was shown that intraperitoneal administration of tamoxifen could cause transient cardiomyopathy in mice. In addition, the dosage was selected was markedly below the 75 mg/kg (maximum dosage) recommended by the Jackson Laboratory to minimize undue cardiotoxicity in the mice. Deletion of Pgm2 in the heart was confirmed by immunoblots on heart tissue using anti-human PGM1 antibody which cross-reacts with the mouse ortholog. Results demonstrated over 95% reduction in Pgm2 protein in Pgm2-cKO hearts (FIG. 2B).

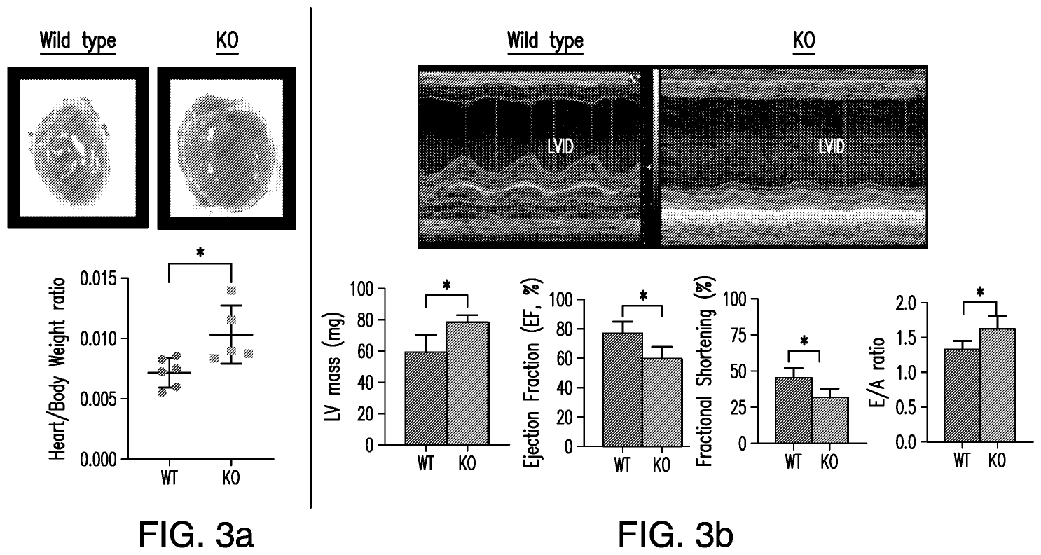

Pgm2 cKO mice develop dilated cardiomyopathy (DCM). At the anatomic level, significantly dilated left ventricles were clearly visible in Pgm2 cKO mice 4 weeks after tamoxifen feeding, while the wild-type mice displayed normal cardiac size (FIG. 3A). Higher lung weights were also noted. Echocardiographic analysis revealed dilated cardiomyopathy in the mutant hearts with decreased fractional shortening (FS), ejection fraction (EF) and increased left ventricular end-diastolic internal dimensions (LVID-d) as early as 2 weeks after tamoxifen feeding, and continue to worsen with age. A representative set of results collected at 12 weeks post-tamoxifen is shown in FIG. 3B. In addition, KO mice also displayed an increase in the early to late ventricular filling velocities (E/A) ratio that was measured across the mitral valve (FIG. 3B).

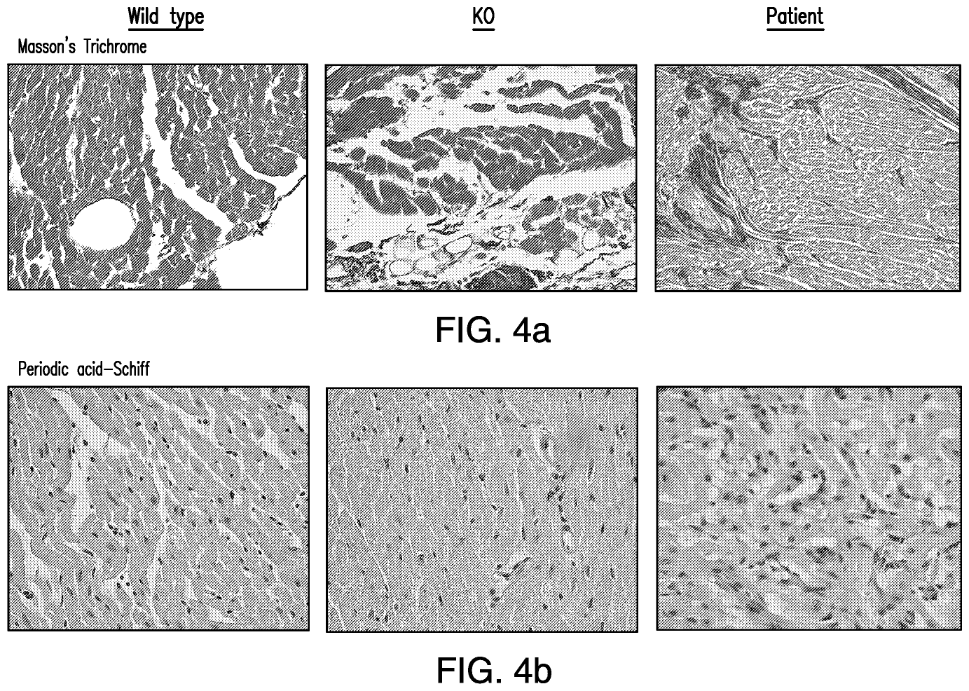

Additional signs of DCM were revealed at the histological levels. At low magnification, hematoxylin and eosin (H & E) staining of WT and Pgm2 cKO hearts as early as 4 weeks after tamoxifen feeding revealed enlarged right and left ventricular chambers with thinner walls. To assess how closely these and latter findings resemble human patient phenotypes, results from a transplanted heart from a young patient with PGM1-CDG was included. Fibrosis was present in the hearts of Pgm2 cKO mice as well as in the heart of PGM1-CDG patient (FIG. 4A). Similarly, hearts from the Pgm2 cKO mice and the PGM1-CDG patient exhibited enhanced glycogen accumulation as evidenced by positive PAS staining (FIG. 4B).

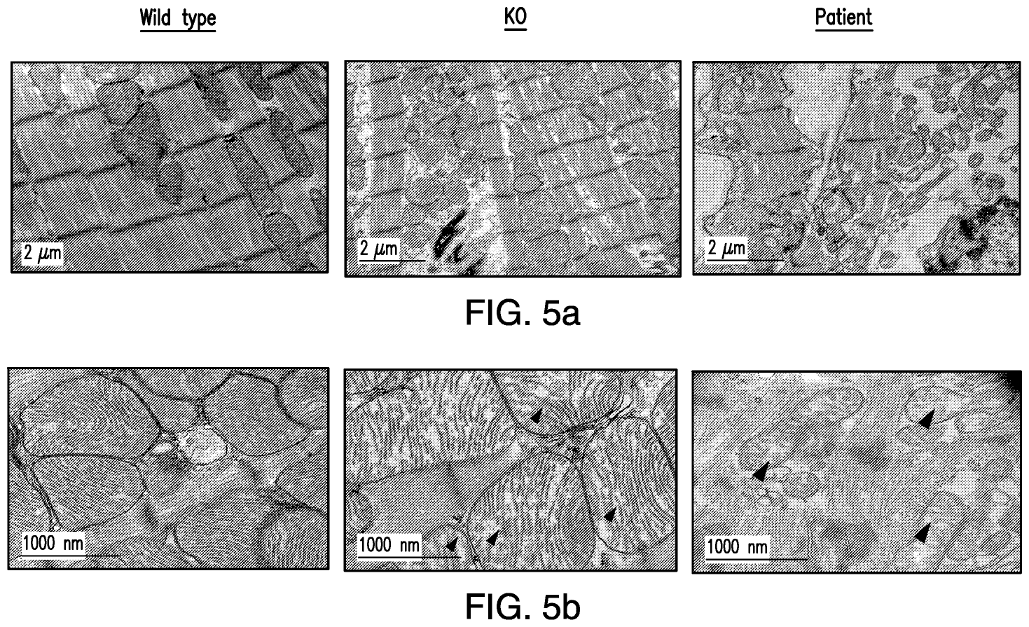

Transmission electron microscopy was used to evaluate the ultrastructure of Pgm2 cKO hearts as well as a heart from a PGM1-CDG patient. As shown in FIG. 5, cardiomyocytes misalignment, reduced mitochondrial matrix density and fragmented mitochondrial cristae in Pgm2 cKO mice was observed. Similarly, loss of cardiomyocyte integrity and abnormal mitochondria with less organized cristae were observed in the heart of the PGM1-CDG patient (FIG. 5).

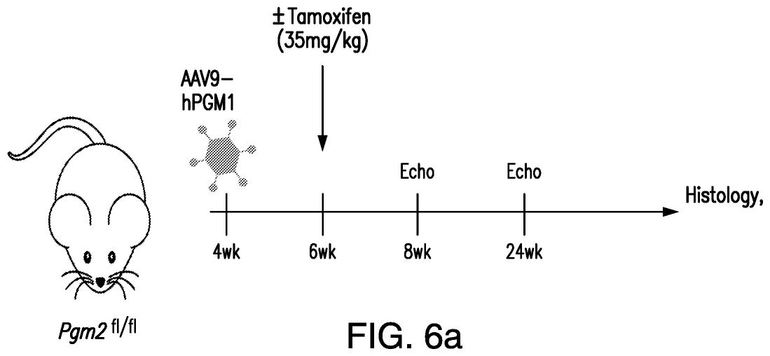

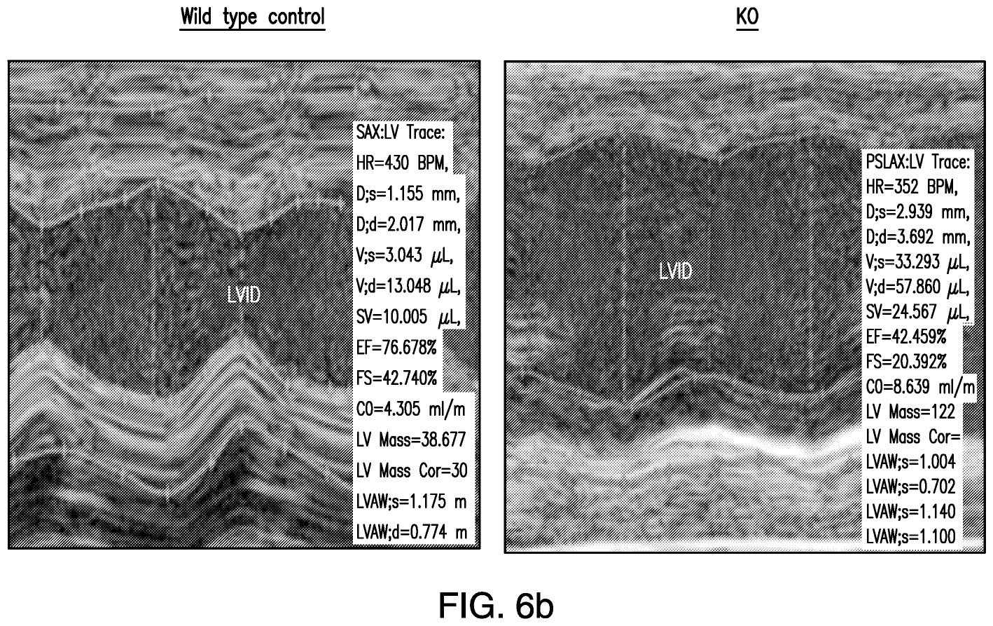

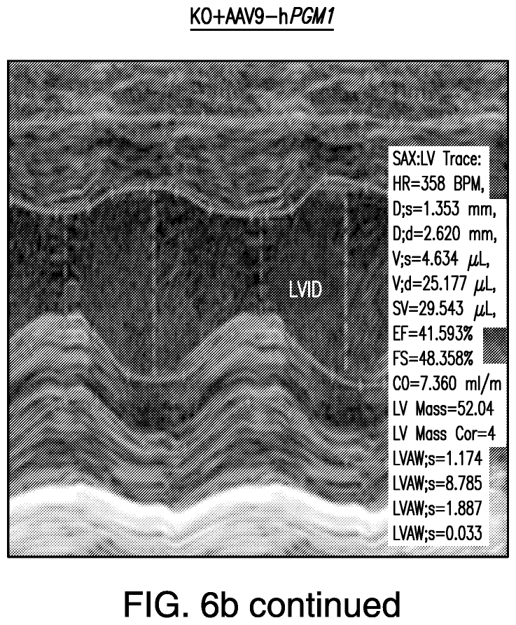

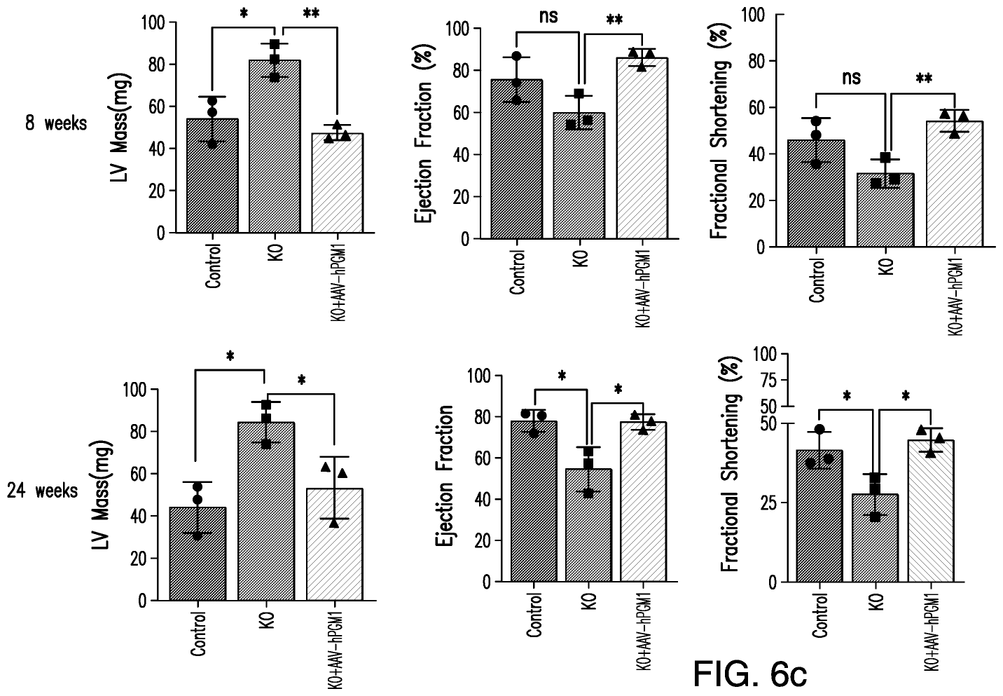

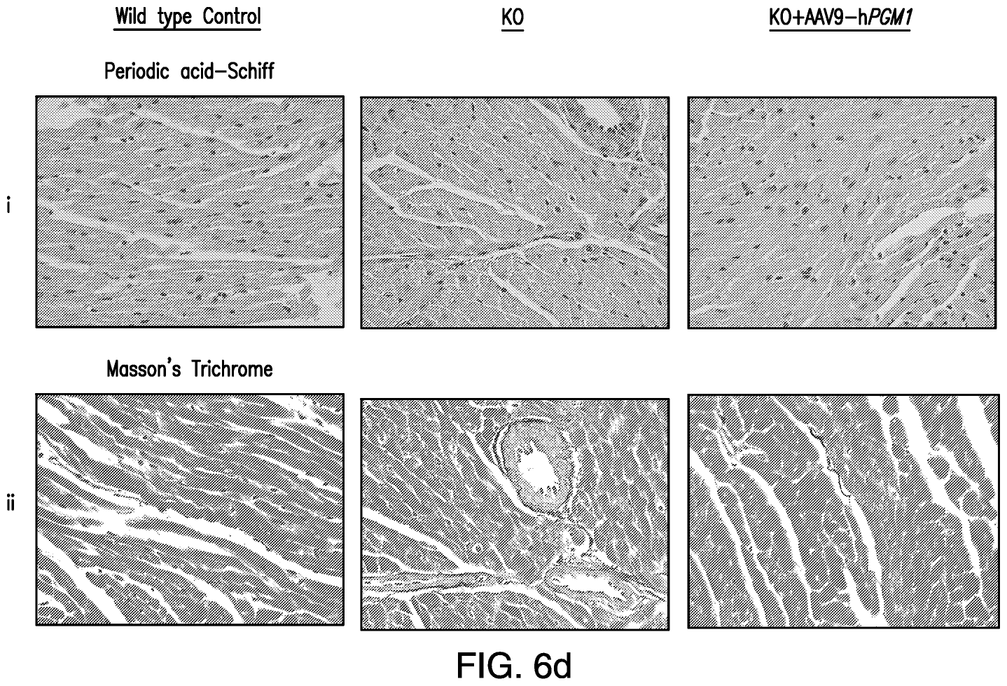

AAV9-hPGM1 gene replacement therapy prevents the manifestation DCM in Pgm2 cKO mice. Three cohorts of age-matched male mice (n=3/4): (1) Pgm2^{flox+/+} Mer-Cre-Mer^{+/-} with AAV gene replacement therapy, (2) Pgm2^{flox+/+} Mer-Cre-Mer^{+/-} without treatment, and Pgm2^{flox-/-} Mer-Cre-Mer^{+/-} without treatment, were used for the experiment. For group (1), 2.5E+13 vg/kg of AAV9-hPGM1 was injected via tail vein at 4 weeks of age. The groups were subjected to tamoxifen feeding two weeks later. Echocardiography was performed at regular time intervals after tamoxifen feeding and the data were compared to untreated Pgm2 cKO and wild type (control, group 3) animals (FIG. 6A).

As shown in FIGS. 6B and 6C, echocardiography of the untreated Pgm2 cKO mice showed significant reduction in ejection fraction (EF) and fractional shortening (FS), increase in LV Mass, and larger left ventricular internal diameter at systole compared to the treated group. The treated group showed no reduction in EF and FS, no increase in LV mass or LV internal diameter over the period of 4 months after treatment. Histological examination of the heart tissues of mice euthanized at the end of 6 months also revealed signs of fibrosis and glycogen accumulation in the untreated KO, but not in the treated and wild-type animals (FIG. 6D).

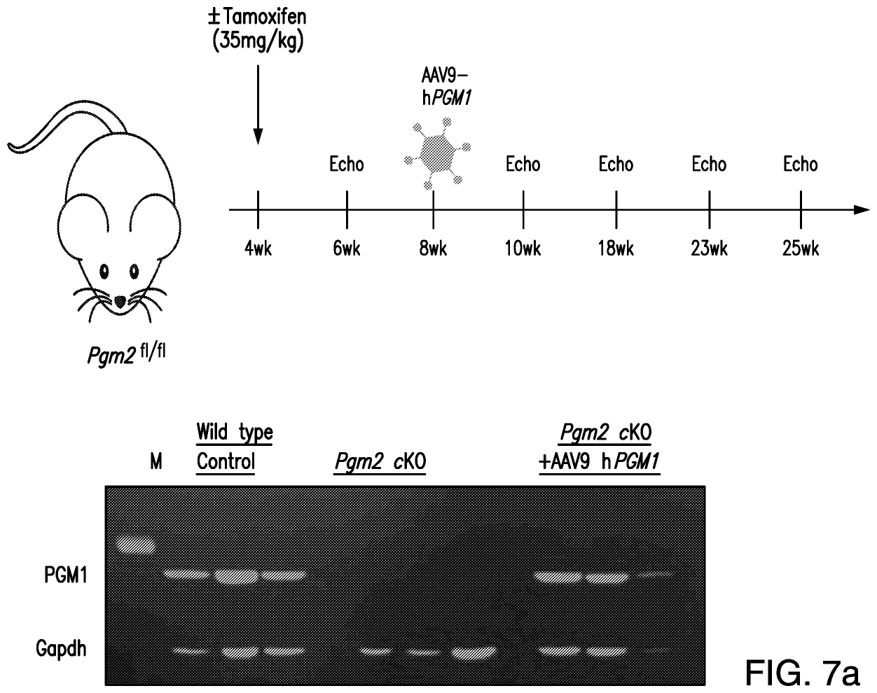

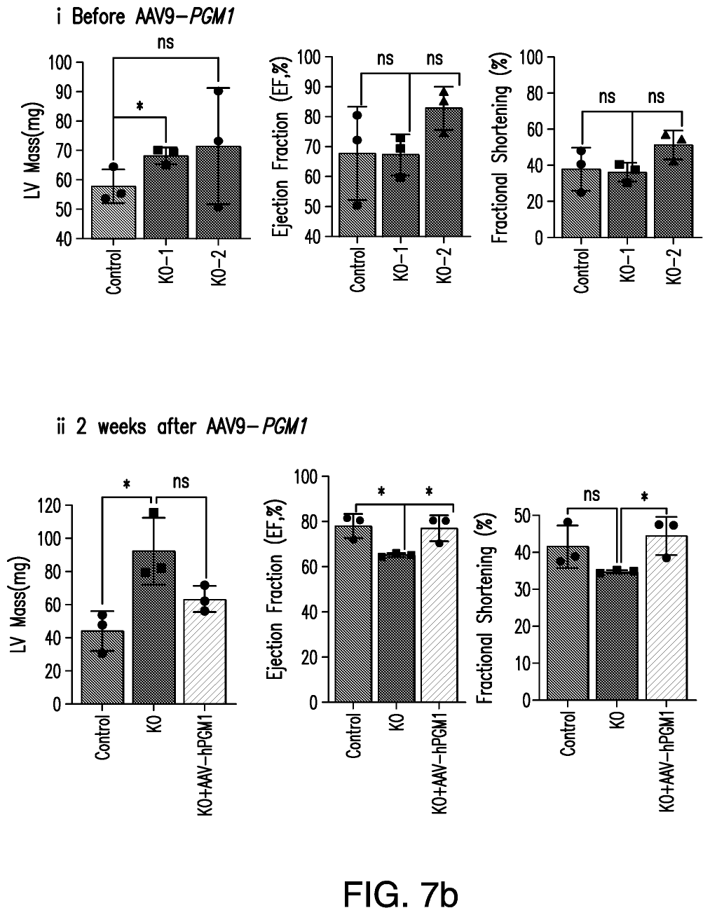

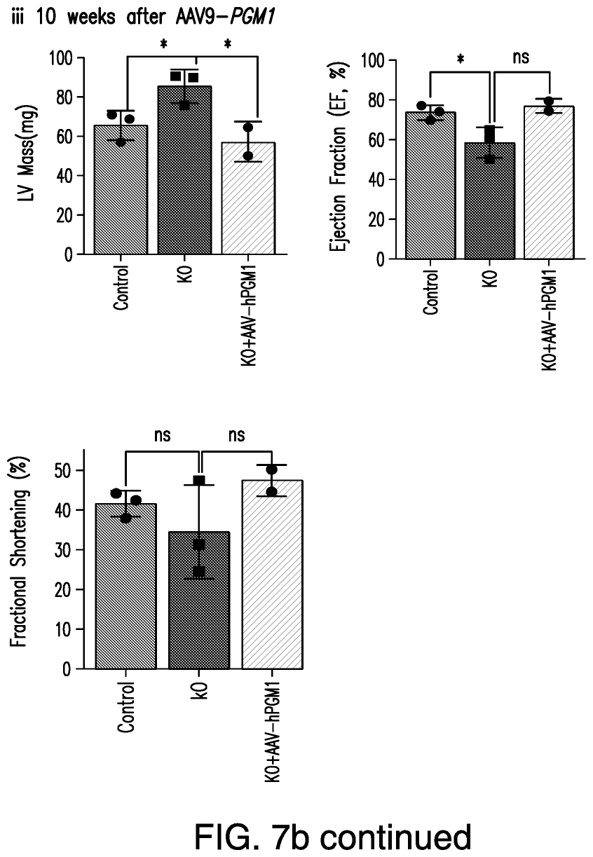

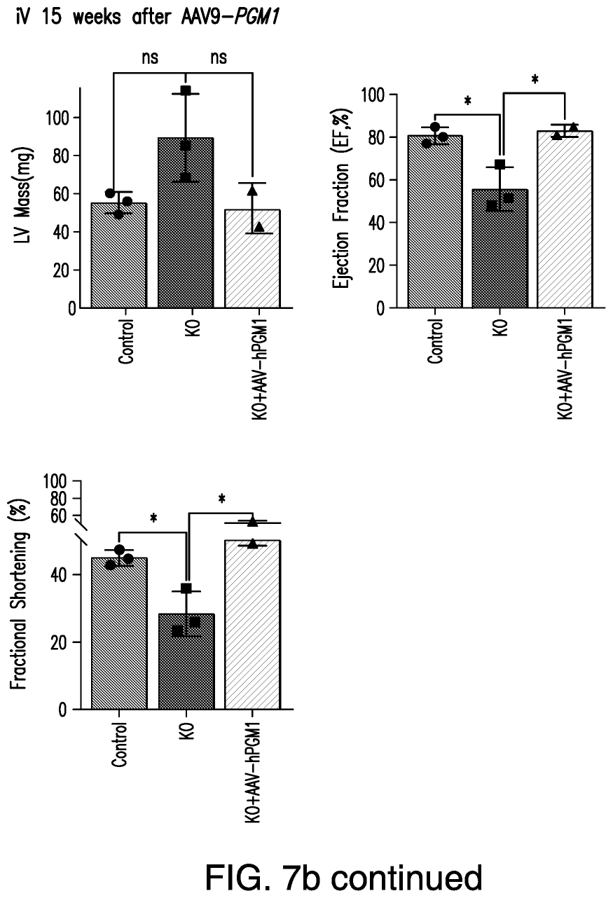

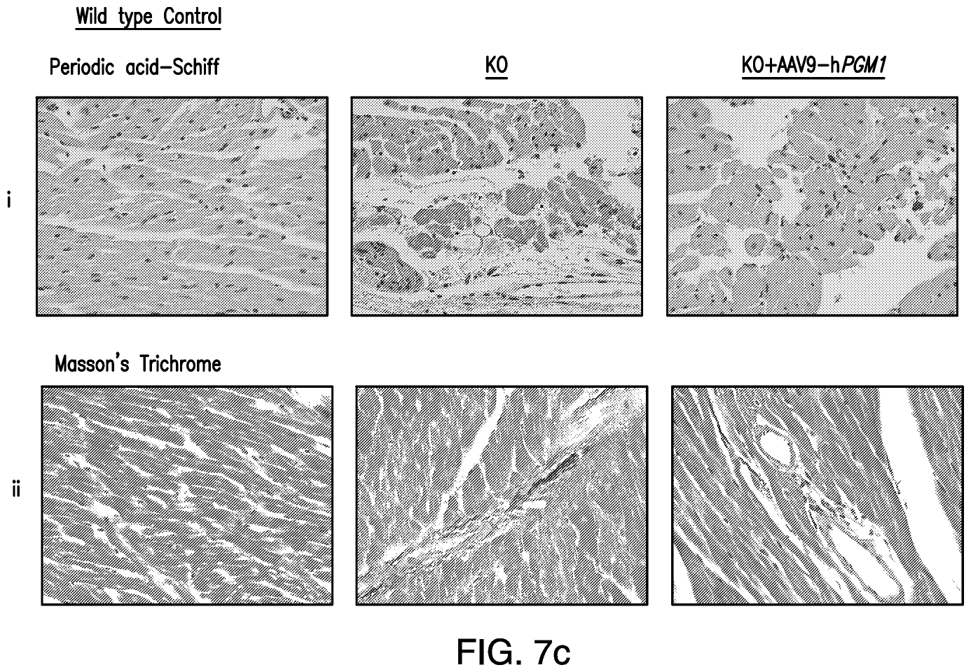

A single intravenous injection of AAV9 hPGM1 halts the progression DCM in Pgm2 cKO mice. To test the efficacy of AAV9-hPGM1 gene replacement therapy in a more clinically relevant context, Pgm2 cKO were induced at 4 weeks of age and echocardiography was performed regularly after until a reduction in cardiac functions was observed. Next, the mice were randomized to treatment among the Pgm2^{flox+/+} Mer-Cre-Mer^{+/-} group with AAV9-hPGM1 and their ECG was monitored for 4 months (FIG. 7A). Hearts were collected at the end point and subjected to histological studies. As shown in FIG. 7B, ECG follow-ups at different time points demonstrated progressive deterioration of cardiac function in the cohort without treatment. However, no further deterioration in cardiac functions was seen the treated group. Such functional studies were corroborated by histological studies shown in FIG. 7C.

What is claimed is:

1. A polynucleotide comprising an expression cassette, wherein the expression cassette comprises a transcriptional regulatory region comprising a promoter operatively linked to a nucleotide sequence as set forth in SEQ ID NO: 1 encoding the human phosphoglucomutase 1 (hPGM1) protein.

2. The polynucleotide of claim 1, wherein the promoter is a constitutive promoter.

3. The polynucleotide of claim 2, wherein the promoter is the CAG, Myh6 or CMV promoter.

4. The polynucleotide of claim 1, wherein the expression cassette is flanked by adeno-associated virus inverted terminal repeats (ITRs).

5. The polynucleotide of claim 2, wherein the CAG promoter has a nucleotide sequence of SEQ ID NO: 2.

6. The polynucleotide of claim 1, further comprising a polyadenylation tail signal.

7. The polynucleotide of claim 6, wherein the polyadenylation tail signal is a bovine growth hormone (bgh) polyadenylation signal

8. The polynucleotide of claim 7, wherein the bgh polyadenylation tail signal has a nucleotide sequence of SEQ ID NO: 3.

9. The polynucleotide of claim 1, further comprising a Kozak sequence.

10. The polynucleotide of claim 9, wherein the Kozak sequence has a nucleotide sequence of SEQ ID NO: 4.

11. The polynucleotide of claim 1, wherein the expression cassette has a nucleic acid sequence of SEQ ID NO: 9.

12. A vector comprising the polynucleotide of claim 1, wherein the vector is an adeno-associated viral vector.

13. The vector of claim 12, wherein the vector is an adeno-associated viral vector of serotype 9 (AAV9).

14. The vector of claim 13, wherein the AAV9 serotype has a capsid that is at least 95% identical to SEQ ID NO: 10 (AAV9 sequence).

15. A pharmaceutical composition comprising a therapeutically effective amount of the vector of claim 12, and a pharmaceutically acceptable carrier and/or adjuvant.

16. A method for the treatment and/or prevention of phosphoglucomutase 1 (PGM1) deficiency in a subject in a subject in need thereof, the method comprising administering to the subject, the pharmaceutical composition of claim 15.

17. A method for the treatment and/or prevention of phosphoglucomutase 1 (PGM1) deficiency in a subject in need thereof, the method comprising administering to the subject, the vector of claim 12.

18. A pharmaceutical composition comprising a therapeutically effective amount of the polynucleotide of claim 1, and a pharmaceutically acceptable carrier and/or adjuvant.

19. A method for the treatment and/or prevention of phosphoglucomutase 1 (PGM1) deficiency in a subject in need thereof comprising administering to the subject, the pharmaceutical composition of claim 18.

20. A method for the treatment and/or prevention of phosphoglucomutase 1 (PGM1) deficiency in a subject in need thereof comprising administering to the subject, the polynucleotide of claim 1.

21. A method for obtaining a recombinant adeno-associated viral vector (AAV) comprising the polynucleotide of claim 1, comprising the steps of: (i) providing a cell comprising the polynucleotide of claim 1, AAV cap proteins, AAV rep proteins and, optionally, viral proteins upon which AAV is dependent for replication, (ii) maintaining the cell under conditions adequate for assembly of the AAV; and (iii) purifying the adeno-associated viral vector produced by the cell.

22. A recombinant adeno-associated virus (AAV) vector comprising an expression cassette comprising: a nucleic acid sequence encoding human phosphoglucomutase 1 (hPGM1), operably linked to one or more regulatory elements; and a polyadenylation tail signal.

23. The recombinant AAV vector of claim 22, wherein the nucleic acid sequence encoding hPGM1 has a nucleic acid sequence of SEQ ID NO: 1.

24. The recombinant AAV vector of claim 22, wherein the nucleic acid sequence encoding hPGM1 comprises a nucleic acid sequence having at least 85% identity to the nucleotide sequence of SEQ ID NO: 1 or a sequence reverse complementary thereto.

25. The recombinant AAV vector of claim 22, wherein the nucleic acid sequence encoding hPGM1 comprises or consisting of the nucleic acid sequence of SEQ ID NO: 1 or a sequence reverse complementary thereto.

26. The recombinant AAV vector of claim 22, wherein the hPGMI protein comprises the amino acid sequence of SEQ ID NO: 11.

27. The recombinant AAV vector of claim 22, wherein the one or more regulatory elements are a CAG promoter and a WPRE sequence.

28. The recombinant AAV vector of claim 27, wherein the CAG promoter has a nucleotide sequence of SEQ ID NO: 2.

29. The recombinant AAV vector of claim 22, wherein the polyadenylation tail signal is a bovine growth hormone (bgh) polyadenylation signal

30. The recombinant AAV vector of claim 29, wherein the bgh polyadenylation tail signal has a nucleotide sequence of SEQ ID NO: 3.

31. The recombinant AAV vector of claim 22, further comprising a Kozak sequence.

32. The recombinant AAV vector of claim 31, wherein the Kozak sequence has a nucleotide sequence of SEQ ID NO: 4.

33. The recombinant AAV vector of claim 22, wherein the expression cassette has a nucleic acid sequence of SEQ ID NO: 9.

34. The recombinant AAV vector of claim 22, wherein the recombinant AAV vector is an AAV9 serotype or has a capsid that is at least 95% identical to SEQ ID NO: 10 (AAV9 sequence).

35. The recombinant AAV vector of claim 22, wherein the nucleic acid sequence encoding hPGM1 is flanked by inverted terminal repeat (ITR) nucleotide sequences.

36. The recombinant AAV vector of claim 35, wherein the ITRs comprise a 5′ ITR having a nucleotide sequence of SEQ ID NO: 5 and a 3′ ITR having a nucleotide sequence of SEQ ID NO: 6, or the reverse complement thereof.

37. The recombinant AAV vector of claim 22, further comprising a selectable marker.

38. A recombinant adeno-associated virus (AAV) vector comprising an AAV9 capsid containing a nucleic acid construct comprising a codon optimized nucleotide sequence encoding human phosphoglucomutase 1 (hPGM1) having the sequence set forth in of SEQ ID NO: 1 operably linked to regulatory elements.

39. The recombinant AAV vector of claim 38, wherein the regulatory elements are a CAG promoter, a WPRE sequence, and a bovine growth hormone (bgh) polyadenylation signal in between AAV-ITR sequences.

40. The recombinant AAV vector of claim 38, wherein the nucleic acid construct has a nucleotide sequence of SEQ ID NO: 9.

41. A pharmaceutical composition comprising the AAV vector of any of claims 22-40.

42. An isolated nucleic acid construct comprising a codon optimized PGM1 encoding nucleotide sequence as set forth by SEQ ID NO: 1 operably linked to regulatory elements for expression of the PGM1 encoding nucleotide sequence in a subject.

43. The isolated nucleic acid construct of claim 42, wherein the nucleic acid construct has the nucleotide sequence of SEQ ID NO: 8 or SEQ ID NO: 9 (construct sequences with and without the ITR sequences).

44. A host cell comprising the isolated nucleic acid constructs of claims 42 or 43.

45. The host cell of claim 44, further comprising an isolated nucleic acid encoding an AAV capsid protein.

46. The host cell of claim 45, wherein the capsid protein is AAV9.

47. A method of producing the recombinant AAV of any of claims 38 to 39 by culturing the host cell of claim 44 or 45.

48. A method of treating a phosphoglucomutase 1 (PGM1) deficiency in a subject in need thereof, the method comprising administering to the subject a therapeutically effective amount of the recombinant AAV vector of any of claims 22-40 or the pharmaceutical composition of claim 41.

49. A method of modulating glycosylation, glucose metabolism, or glycogen metabolism in a subject, the method comprising administering to the subject a therapeutically effective amount of the recombinant AAV vector of any of claims 22-40 or the pharmaceutical composition of claim 41.

50. A method of increasing ejection fraction or fractional shortening in a subject, the method comprising administering to the subject a therapeutically effective amount of the recombinant AAV vector of any of claims 22-40 or the pharmaceutical composition of claim 41.

51. A method of reducing left ventricular mass in a subject, the method comprising administering to the subject a therapeutically effective amount of the recombinant AAV vector of any of claims 22-40 or the pharmaceutical composition of claim 41.

52. A method of reducing the early (E) to late (A) ventricular filing velocities (E/A ratio) in a subject, the method comprising administering to the subject a therapeutically effective amount of the recombinant AAV vector of any of claims 22-40 or the pharmaceutical composition of claim 41.

53. A method of reducing a disease condition in a subject suffering from PGM1-CDG, the method comprising administering to the subject a therapeutically effective amount of the recombinant AAV vector of any of claims 22-40 or the pharmaceutical composition of claim 41, wherein the disease condition is hyptonia, hypoglycemia, cardiomyopathy, growth retardation, hormonal deficiencies, myopathy, hypogonadotropic hypogonadism, malignant hyperthermia, coagulation disorders or hepatopathy.

54. The method of any of claims 48-53, wherein the subject has a PGM1 deficiency or has been diagnosed with PGM1-CDG.

55. The method of claims 48-54, wherein the administering is intravenous, intramuscular, or intracardiac.


SEQUENCE LISTING (13 sequences)

SEQ ID NO: 1 - DNA, 1689 bp, Artificial Sequence
  Synthetic construct
atggtgaagatcgtgacagttaagacccaggcgtaccaggaccagaagccgggcacgagcgggctgcggaagcgggtgaaggtgttccagagcagcgccaactacgcggagaacttcatccagagtatcatctccaccgtggagccggcgcagcggcaggaggccacgctggtggtgggcggggacggccggttctacatgaaggaggccatccagctcatcgctcgcatcgctgccgccaacgggatcggtcgcttggttatcggacagaatggaatcctctccacccctgctgtatcctgcatcattagaaaaatcaaagccattggtgggatcattctgacagccagtcacaacccagggggccccaatggagattttggaatcaaattcaatatttctaatggaggtcctgctccagaagcaataactgataaaattttccaaatcagcaagacaattgaagaatatgcagtttgccctgacctgaaagtagaccttggtgttctgggaaagcagcagtttgacttggaaaataagttcaaacccttcacagtggaaattgtggattcggtagaagcttatgctacaatgctgagaagcatctttgatttcagtgcactgaaagaactactttctgggccaaaccgactgaagatccgtattgatgctatgcatggagttgtgggaccgtatgtaaagaagatcctctgtgaagaactcggtgcccctgcgaactcggcagttaactgcgttcctctggaggactttggaggccaccaccctgaccccaacctcacctatgcagctgacctggtggagaccatgaagtcaggagagcatgattttggggctgcctttgatggagatggggatcgaaacatgattctgggcaagcatgggttctttgtgaacccttcagactctgtggctgtcattgctgccaacatcttcagcattccgtatttccagcagactggggtccgcggctttgcacggagcatgcccacgagtggtgctctggaccgggtggctagtgctacaaagattgctttgtatgagaccccaactggctggaagttttttgggaatttgatggacgcgagcaaactgtccctttgtggggaggagagcttcgggaccggttctgaccacatccgtgagaaagatggactgtgggctgtccttgcctggctctccatcctagccacccgcaagcagagtgtggaggacattctcaaagatcattggcaaaagtatggccggaatttcttcaccaggtatgattacgaggaggtggaagctgagggcgcaaacaaaatgatgaaggacttggaggccctgatgtttgatcgctcctttgtggggaagcagttctcagcaaatgacaaagtttacactgtggagaaggccgataactttgaatacagcgacccagtggatggaagcatttcaagaaatcagggcttgcgcctcattttcacagatggttctcgaatcgtcttccgactgagcggcactgggagtgccggggccaccattcggctgtacatcgatagctatgagaaggacgttgccaagattaaccaggacccccaggtcatgttggccccccttatttccattgctctgaaagtgtcccagctgcaggagaggacgggacgcactgcacccactgtcatcacctaa

SEQ ID NO: 2 - DNA, 1733 bp, Artificial Sequence
  Synthetic construct
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattg

SEQ ID NO: 3 - DNA, 208 bp, Artificial Sequence
  Synthetic construct
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 4 - DNA, 6 bp, Artificial Sequence
  Synthetic construct
gccacc

SEQ ID NO: 5 - DNA, 141 bp, Artificial Sequence
  Synthetic construct
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 6 - DNA, 141 bp, Artificial Sequence
  Synthetic construct
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 7 - DNA, 598 bp, Artificial Sequence
  Synthetic construct
cgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgg

SEQ ID NO: 8 - DNA, 4634 bp, Artificial Sequence
  Synthetic construct
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatggtgaagatcgtgacagttaagacccaggcgtaccaggaccagaagccgggcacgagcgggctgcggaagcgggtgaaggtgttccagagcagcgccaactacgcggagaacttcatccagagtatcatctccaccgtggagccggcgcagcggcaggaggccacgctggtggtgggcggggacggccggttctacatgaaggaggccatccagctcatcgctcgcatcgctgccgccaacgggatcggtcgcttggttatcggacagaatggaatcctctccacccctgctgtatcctgcatcattagaaaaatcaaagccattggtgggatcattctgacagccagtcacaacccagggggccccaatggagattttggaatcaaattcaatatttctaatggaggtcctgctccagaagcaataactgataaaattttccaaatcagcaagacaattgaagaatatgcagtttgccctgacctgaaagtagaccttggtgttctgggaaagcagcagtttgacttggaaaataagttcaaacccttcacagtggaaattgtggattcggtagaagcttatgctacaatgctgagaagcatctttgatttcagtgcactgaaagaactactttctgggccaaaccgactgaagatccgtattgatgctatgcatggagttgtgggaccgtatgtaaagaagatcctctgtgaagaactcggtgcccctgcgaactcggcagttaactgcgttcctctggaggactttggaggccaccaccctgaccccaacctcacctatgcagctgacctggtggagaccatgaagtcaggagagcatgattttggggctgcctttgatggagatggggatcgaaacatgattctgggcaagcatgggttctttgtgaacccttcagactctgtggctgtcattgctgccaacatcttcagcattccgtatttccagcagactggggtccgcggctttgcacggagcatgcccacgagtggtgctctggaccgggtggctagtgctacaaagattgctttgtatgagaccccaactggctggaagttttttgggaatttgatggacgcgagcaaactgtccctttgtggggaggagagcttcgggaccggttctgaccacatccgtgagaaagatggactgtgggctgtccttgcctggctctccatcctagccacccgcaagcagagtgtggaggacattctcaaagatcattggcaaaagtatggccggaatttcttcaccaggtatgattacgaggaggtggaagctgagggcgcaaacaaaatgatgaaggacttggaggccctgatgtttgatcgctcctttgtggggaagcagttctcagcaaatgacaaagtttacactgtggagaaggccgataactttgaatacagcgacccagtggatggaagcatttcaagaaatcagggcttgcgcctcattttcacagatggttctcgaatcgtcttccgactgagcggcactgggagtgccggggccaccattcggctgtacatcgatagctatgagaaggacgttgccaagattaaccaggacccccaggtcatgttggccccccttatttccattgctctgaaagtgtcccagctgcaggagaggacgggacgcactgcacccactgtcatcacctaaacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 9 - DNA, 5409 bp, Artificial Sequence
  Synthetic construct
tctagacaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatggtgaagatcgtgacagttaagacccaggcgtaccaggaccagaagccgggcacgagcgggctgcggaagcgggtgaaggtgttccagagcagcgccaactacgcggagaacttcatccagagtatcatctccaccgtggagccggcgcagcggcaggaggccacgctggtggtgggcggggacggccggttctacatgaaggaggccatccagctcatcgctcgcatcgctgccgccaacgggatcggtcgcttggttatcggacagaatggaatcctctccacccctgctgtatcctgcatcattagaaaaatcaaagccattggtgggatcattctgacagccagtcacaacccagggggccccaatggagattttggaatcaaattcaatatttctaatggaggtcctgctccagaagcaataactgataaaattttccaaatcagcaagacaattgaagaatatgcagtttgccctgacctgaaagtagaccttggtgttctgggaaagcagcagtttgacttggaaaataagttcaaacccttcacagtggaaattgtggattcggtagaagcttatgctacaatgctgagaagcatctttgatttcagtgcactgaaagaactactttctgggccaaaccgactgaagatccgtattgatgctatgcatggagttgtgggaccgtatgtaaagaagatcctctgtgaagaactcggtgcccctgcgaactcggcagttaactgcgttcctctggaggactttggaggccaccaccctgaccccaacctcacctatgcagctgacctggtggagaccatgaagtcaggagagcatgattttggggctgcctttgatggagatggggatcgaaacatgattctgggcaagcatgggttctttgtgaacccttcagactctgtggctgtcattgctgccaacatcttcagcattccgtatttccagcagactggggtccgcggctttgcacggagcatgcccacgagtggtgctctggaccgggtggctagtgctacaaagattgctttgtatgagaccccaactggctggaagttttttgggaatttgatggacgcgagcaaactgtccctttgtggggaggagagcttcgggaccggttctgaccacatccgtgagaaagatggactgtgggctgtccttgcctggctctccatcctagccacccgcaagcagagtgtggaggacattctcaaagatcattggcaaaagtatggccggaatttcttcaccaggtatgattacgaggaggtggaagctgagggcgcaaacaaaatgatgaaggacttggaggccctgatgtttgatcgctcctttgtggggaagcagttctcagcaaatgacaaagtttacactgtggagaaggccgataactttgaatacagcgacccagtggatggaagcatttcaagaaatcagggcttgcgcctcattttcacagatggttctcgaatcgtcttccgactgagcggcactgggagtgccggggccaccattcggctgtacatcgatagctatgagaaggacgttgccaagattaaccaggacccccaggtcatgttggccccccttatttccattgctctgaaagtgtcccagctgcaggagaggacgggacgcactgcacccactgtcatcacctaaacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagt

SEQ ID NO: 10 - protein, 736 aa, Artificial Sequence
  Synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 11 - protein, 562 aa, Artificial Sequence
  Synthetic construct
MVKIVTVKTQAYQDQKPGTSGLRKRVKVFQSSANYAENFIQSIISTVEPAQRQEATLVVGGDGRFYMKEAIQLIARIAAANGIGRLVIGQNGILSTPAVSCIIRKIKAIGGIILTASHNPGGPNGDFGIKFNISNGGPAPEAITDKIFQISKTIEEYAVCPDLKVDLGVLGKQQFDLENKFKPFTVEIVDSVEAYATMLRSIFDFSALKELLSGPNRLKIRIDAMHGVVGPYVKKILCEELGAPANSAVNCVPLEDFGGHHPDPNLTYAADLVETMKSGEHDFGAAFDGDGDRNMILGKHGFFVNPSDSVAVIAANIFSIPYFQQTGVRGFARSMPTSGALDRVASATKIALYETPTGWKFFGNLMDASKLSLCGEESFGTGSDHIREKDGLWAVLAWLSILATRKQSVEDILKDHWQKYGRNFFTRYDYEEVEAEGANKMMKDLEALMFDRSFVGKQFSANDKVYTVEKADNFEYSDPVDGSISRNQGLRLIFTDGSRIVFRLSGTGSAGATIRLYIDSYEKDVAKINQDPQVMLAPLISIALKVSQLQERTGRTAPTVIT

SEQ ID NO: 12 - DNA, 20 bp, Artificial Sequence
  Synthetic construct
tgtgacagtccatctgggga

SEQ ID NO: 13 - DNA, 20 bp, Artificial Sequence
  Synthetic construct
cttcgggacccgagactaag